(19) United States
(12) Patent Application Publication
(10) Pub. No.: US 2023/0142780 A1
(43) Pub. Date: May 11, 2023

(54) USE OF VIRAL VECTORS FOR CORONAVIRUS VACCINE PRODUCTION

(71) Applicants: 4MVac LLC, Norristown, PA (US); Trustees of the University of Pennsylvania, Philadelphia, PA (US); Johnson & Johnson, Inc., New Brunswick, PA (US)

(72) Inventors: Hansell Hall Stedman, Norristown, PA (US); Geoffrey Tabin, Portola Valley, CA (US); Charles R. Bridges, Newton, MA (US)

(73) Assignees: 4MVac LLC, Norristown, PA (US); Trustees of the University of Pennsylvania, Philadelphia, PA (US); Johnson & Johnson, Inc., New Brunswick, NJ (US)

(21) Appl. No.: 17/918,000

(22) PCT Filed: Apr. 9, 2021
(86) PCT No.: PCT/US2021/026566

§ 371 Date: Oct. 10, 2022

Related U.S. Application Data
(60) Provisional application No. 63/007,865, filed on Apr. 9, 2020.

(51) Int. Cl. A61K 39/215; C12N 15/86; C07K 14/005

(52) U.S. Cl. CPC A61K 39/215; C12N 15/86; C07K 14/005; C12N 2750/14143; A61K 2039/57

(57) ABSTRACT

Provided herein are compositions that includes AAVs and AAV vectors that include a sequence encoding a SARS-CoV-2 polypeptide or a fragment thereof. Also provided herein are methods and materials for making and using AAVs and AAV vectors to generate immunity to a coronavirus in a subject.

### CROSS-REFERENCE TO RELATED APPLICATIONS

This application claims priority to U.S. Provisional Patent Application No. 63/007,865, filed Apr. 9, 2020, which is incorporated herein by reference in its entirety.

### TECHNICAL FIELD

The present disclosure relates generally to the use of adeno-associated virus to generate immunity to a coronavirus in a subject.

### BACKGROUND OF THE INVENTION

Coronaviruses (CoVs) are enveloped RNA viruses that are characterized by club-like spikes that project from their surface, a unique replication strategy, and a large RNA genome (Fehr and Perlman, Coronaviruses, 1282:1-23, 2015). Coronaviruses cause a variety of diseases in mammals and birds ranging from enteritis in cows and pigs, and upper respiratory disease in chickens to potentially lethal human respiratory infections.

Severe acute respiratory syndrome (SARS) is a newly emerging infectious disease caused by a coronavirus, which includes SARS-coronavirus (SARS-CoV) and SARS-coronavirus-2 (SARS-CoV-2). The SARS-CoV and SARS-CoV-2 each include a spike (S) protein is composed of two subunits. The S1 subunit contains a receptor-binding domain that engages with the host cell receptor angiotensin-converting enzyme 2 and the S2 subunit mediates fusion between the viral and host cell membranes. The S protein plays a key part in the induction of humoral immunity (e.g., neutralizing-antibody) and cellular immunity (e.g., T-cell responses), as well as protective immunity, during infection with SARS-CoV and SARS-CoV-2. Given the emergence of SARS-CoV-2 (COVID-19), there remains a critical unmet need for a vaccine against coronaviruses. However, current vaccination strategies do not impart the necessary cellular immunity needed for a full immunogenic response. Therefore, there remains a critical unmet need to develop a vaccine against coronaviruses that will produce robust, sustained cellular immunity (T-cell responses).

### SUMMARY

This document provides methods and materials for making and using AAVs and vectors to generate immunity to a coronavirus in a subject. For example, this document provides AAVs or vectors containing a sequence encoding a SARS-CoV-2 S glycoprotein polypeptide or a fragment thereof. In another example, this document provides AAVs or vectors containing sequence encoding a SARS-CoV-2 nucleocapsid polypeptide or fragment thereof. Such an AAV can be used to produce a novel vaccine against coronaviruses, including SARS-CoV-2. The immunogenicity induced by the AAV (or vector) containing a sequence encoding either a SARS-CoV-2 S glycoprotein polypeptide or fragment thereof or a SARS-CoV-2 nucleocapsid polypeptide or fragment thereof employs both cellular (e.g., T-cell mediated) and humoral (e.g., neutralizing antibodies) immune responses against the SARS-CoV-2 polypeptide in the subject, thereby creating at least partial immunity to the coronavirus.

In one aspect, this disclosure features adeno-associated virus (AAV) vectors including a sequence encoding a SARS-CoV-2 polypeptide or a fragment thereof.

In some embodiments, the SARS-CoV-2 polypeptide is a SARS-CoV-2 S glycoprotein polypeptide or a fragment thereof.

In some embodiments, the S glycoprotein polypeptide or the fragment thereof includes a S1 domain or a fragment thereof.

In some embodiments, the S glycoprotein polypeptide or the fragment thereof includes, consists essentially, or consists of a S2 domain or a fragment thereof. In some embodiments, the S2 domain or the fragment thereof includes, consists essentially, or consists of SEQ ID NO: 2 or SEQ ID NO: 3.

In some embodiments, the AAV also includes a sequence encoding a transmembrane domain, wherein the sequence encoding the transmembrane domain is operably linked to the sequence encoding the S glycoprotein polypeptide or the fragment thereof. In some embodiments, the transmembrane domain includes a sequence of SEQ ID NO: 4 or a fragment thereof.

In some embodiments, the SARS-CoV-2 polypeptide or the fragment thereof is a nucleocapsid protein or a fragment thereof. In some embodiments, the nucleocapsid polypeptide or the fragment thereof includes a sequence of SEQ ID NO: 5 or a fragment thereof.

In some embodiments, the SARS-CoV-2 polypeptide or the fragment thereof is an envelope polypeptide or a fragment thereof.

In some embodiments, the SARS-CoV-2 polypeptide or the fragment thereof is a membrane polypeptide or a fragment thereof.

In some embodiments, the AAV vectors also includes a sequence encoding a signal sequence, wherein the sequence encoding the signal sequence is operatively linked to the sequence encoding the SARS CoV-2 polypeptide or the fragment thereof. In some embodiments, the signal sequence includes a sequence of SEQ ID NO: 6.

In some embodiments, the AAV also includes a promoter operatively linked to the sequence encoding the SARS CoV-2 polypeptide or the fragment thereof. In some embodiments, the promoter is a muscle-specific promoter. In some embodiments, the muscle-specific promoter is selected from the group consisting of: skeletal β-actin, myosin light chain 2A, dystrophin, muscle creatine kinase, SPc-512, and synthetic muscle promoters. In some embodiments, the promoter is selected from the group consisting of: CMV, RSV, SV40, β-actin, PGK, and EF1 promoters.

In some embodiments, the AAV vector includes an AAV serotype 6 (AAV6) capsid protein. In some embodiments, the AAV vector includes an AAV serotype 9 (AAV9) capsid protein. In some embodiments, the AAV vector includes an Anc80, Anc80Lib, Anc 81, Anc82, Anc83, Anc84, Anc110, Anc113, Anc126, Anc127, or another phylogenetically related AAV capsid protein.

In another aspect, this disclosure features pharmaceutical compositions including any of the AAV vectors described herein. In some embodiments, the pharmaceutical composition also includes an adjuvant. In some embodiments, the adjuvant is a CpG adjuvant.

In another aspect, this disclosure features vaccine compositions that include any of the AAV vectors described herein. In some embodiments, the vaccine composition also includes an adjuvant. In some embodiments, the adjuvant is a CpG adjuvant.

In another aspect, this disclosure features vectors including a sequence encoding a SARS-CoV-2 polypeptide or a fragment thereof.

In some embodiments, the SARS-CoV-2 polypeptide is a SARS-CoV-2 S glycoprotein polypeptide or a fragment thereof.

In some embodiments, the S glycoprotein polypeptide or the fragment thereof includes a S1 domain or a fragment thereof.

In some embodiments, the S glycoprotein polypeptide or the fragment thereof includes, consists essentially, or consists of a S2 domain or a fragment thereof. In some embodiments, the S2 domain or the fragment thereof includes, consists essentially, or consists of SEQ ID NO: 2 or SEQ ID NO: 3.

In some embodiments, the vector also includes a sequence encoding a transmembrane domain, wherein the sequence encoding the transmembrane domain is operably linked to the sequence encoding the S glycoprotein polypeptide or the fragment thereof. In some embodiments, the transmembrane domain includes a sequence of SEQ ID NO: 4 or a fragment thereof.

In some embodiments, the SARS-CoV-2 polypeptide or the fragment thereof is a nucleocapsid protein or a fragment thereof. In some embodiments, the nucleocapsid polypeptide or the fragment thereof includes a sequence of SEQ ID NO: 5 or a fragment thereof.

In some embodiments, the SARS-CoV-2 polypeptide or the fragment thereof is an envelope polypeptide or a fragment thereof.

In some embodiments, the SARS-CoV-2 polypeptide or the fragment thereof is a membrane polypeptide or a fragment thereof.

In some embodiments, the vector also includes a sequence encoding a signal sequence, wherein the sequence encoding the signal sequence is operatively linked to the sequence encoding the SARS CoV-2 polypeptide or the fragment thereof. In some embodiments, the signal sequence includes a sequence of SEQ ID NO: 6.

In some embodiments, the vector further includes a promoter operatively linked to the sequence encoding the SARS CoV-2 polypeptide or the fragment thereof. In some embodiments, the promoter is a muscle-specific promoter. In some embodiments, the muscle-specific promoter is selected from the group consisting of: skeletal β-actin, myosin light chain 2A, dystrophin, muscle creatine kinase, SPc-512, and synthetic muscle promoters. In some embodiments, the promoter is selected from the group consisting of: CMV, RSV, SV40, β-actin, PGK, and EF1 promoters.

In some embodiments, the vector is a viral vector. In some embodiments, the viral vector is a lentivirus vector or herpes virus vector.

In some embodiments, the vector is a plasmid.

In another aspect, this disclosure features pharmaceutical compositions including any of the vectors described herein. In some embodiments, the pharmaceutical composition also includes an adjuvant. In some embodiments, the adjuvant is a CpG adjuvant.

In another aspect, this disclosure features vaccine compositions including any of the vectors described herein. In some embodiments, the vaccine compositions also include an adjuvant. In some embodiments, the adjuvant is a CpG adjuvant.

In another aspect, this disclosure features methods of inducing at least partial immunity to a coronavirus in a subject, the method including administering to the subject a therapeutically effective amount of any of the AAV or AAV vectors described herein, any of the vectors described herein, any of the pharmaceutical compositions described herein, or any of the vaccine compositions described herein.

In some embodiments, the administering results in at least partial immunity to the coronavirus due to humoral immunity to the coronavirus.

In some embodiments, the administering results in robust T-cell mediated immunity to the coronavirus.

In some embodiments, the administering results in an increase in titer of antibodies that specifically bind to the SARS-CoV-2 polypeptide or the fragment thereof in the subject.

In some embodiments, the administering results in a decrease in the rate of infection of the coronavirus in the subject.

In some embodiments, the method further includes administering an adjuvant to the subject. In some embodiments, the adjuvant is a CpG adjuvant.

In some embodiments, the subject has been identified as not having previously had a coronavirus infection.

In some embodiments, prior to the administering step, the subject has been identified as not having a significant titer of antibodies that bind specifically to the SARS-CoV-2 polypeptide of the fragment thereof.

In some embodiments, the coronavirus is SARS-CoV-2.

In some embodiments, the subject has been previously identified as having one or more medical conditions selected from the group consisting of: chronic lung disease, moderate asthma, severe asthma, heart conditions, diabetes, obesity, liver disease, chronic kidney disease, and a weakened or suppressed immune system.

In some embodiments, the subject having a weakened or suppressed immune system is a subject receiving a cancer treatment, a smoker, a subject who is a transplant recipient, a subject having HIV or AIDS, or a subject receiving a corticosteroid or any other immunosuppressant drug.

In some embodiments, the subject having a weakened or suppressed immune system is an elderly subject.

The term “a” and “an” refers to one or to more than one (i.e., at least one) of the grammatical object of the article. By way of example, “an element” encompasses one element and more than one element.

The term “subject” refers to humans and also to non-human mammals such as, but not limited to, non-human primates, cats, dogs, sheep, goats, horses, cows, pigs and rodents, such as but not limited to, mice and rats; as well as to non-mammalian animals such as, but not limited to, birds, poultry, reptiles, amphibians.

Unless otherwise specified, a “nucleotide sequence encoding an amino acid sequence” includes all nucleotide sequences that are degenerate versions of each other and thus encode the same amino acid sequence.

The term “endogenous” refers to any material originating from within an organism, cell, or tissue.

The term “exogenous” refers to any material introduced from or originating from outside an organism, cell, or tissue that is not produced or does not originate from the same organism, cell, or tissue in which it is being introduced.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

The term “transfected,” “transformed,” or “transduced” refers to a process by which exogenous nucleic acid is transferred or introduced into a cell. A “transfected,” “transformed,” or “transduced” mammalian cell is one that has been transfected, transformed or transduced with exogenous nucleic acid.

The term “expression” refers to the transcription and/or translation of a particular nucleotide sequence encoding a protein.

The term “transient expression” refers to the expression of a non-integrated coding sequence for a short period of time (e.g., hours or days). The coding sequence that is transiently expressed in a cell is lost upon multiple rounds of cell division.

A treatment is “therapeutically effective” when it results in at least partial immunity to a coronavirus in a subject. A “therapeutically effective” treatment can also refer to treatment that results in a reduction in one or more of the number, severity, and frequency of one or more symptoms of a disease state (e.g. COVID-19) in a subject.

The term “nucleic acid” or “polynucleotide” refers to deoxyribonucleic acid (DNA) or ribonucleic acid (RNA), or a combination thereof, in either single- or double-stranded form. Unless specifically limited, the term encompasses nucleic acids containing known analogues of natural nucleotides that have similar binding properties as the reference nucleotides. Unless otherwise indicated, a particular nucleic acid sequence also implicitly encompasses complementary sequences as well as the sequence explicitly indicated. In some embodiments of any of the nucleic acids described herein, the nucleic acid is DNA. In some embodiments of any of the nucleic acids described herein, the nucleic acid is RNA.

Unless otherwise defined, all technical and scientific terms used herein have the same meaning as commonly understood by one of ordinary skill in the art to which this invention belongs. Methods and materials are described herein for use in the present invention; other suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### BRIEF DESCRIPTION OF DRAWINGS

FIG. 1A is the amino acid sequence of SARS-CoV-2 S glycoprotein corresponding to SEQ ID NO: 1. The amino acid sequences corresponding to exemplary domains are provided below. Signal sequence (SS) indicated with a box around the amino acid sequence and an “SS.” N-terminal domain (NTD) of S1 indicated with underlined text. Receptor binding domain (RBD) indicated with italicized text. S1/S2 protease cleavage site (S1/S2) indicated with a box around the amino acid sequence and an “S1/S2”. S2′ protease cleavage site (S2) indicated with underlined text surrounded by double brackets. Fusion peptide (FP) indicated with bold text. Heptad repeat 1 (HR1) indicated with italicized text surrounded by double brackets. Central helix (CH) indicated with bold text surrounded by double brackets. Connector domain (CD) indicated as underlined and italicized text surrounded by double brackets. Transmembrane domain (TM) indicated as underlined and bolded text surrounded by double brackets.

FIG. 1B is an exemplary schematic showing the domains of the SARS-CoV-2 S glycoprotein (amino acids 1 to 1273). SS—signal sequence. NTD—N-terminal domain of S1. RBD—receptor binding domain. SD1—subdomain 1. SD2—subdomain 2. S1/S2 protease cleavage site (indicated with arrow and text). S2′ protease cleavage site (indicated with arrow and text). (FP) Fusion peptide. HR1—Heptad repeat 1. CH—central helix. CD—connector domain. HR2—heptad repeat 2. TM—transmembrane domain. CT—cytoplasmic tail. Residue 1208 indicates the end of the ectoderm domain of the SARS-CoV-2 S glycoprotein.

FIG. 2 is a schematic of a rAAV plasmid encoding full length SARS-CoV-2 S glycoprotein and corresponding structures of the S1 domain and S2 domain polypeptides. Inset box shows a schematic of transcription factor binding sites located in the CMV promoter sequence. SRE—serum response element. NFkB—binding site for nuclear factor-κB family of transcription factors. Sp1—binding site for Sp1 transcription factor. STAT—binding site for signal transducer and activator of transcription (STAT) family of transcription factors. AP-1—binding site for activator protein 1 (AP-1) transcription factor. CREB—is a binding site for cAMP response element-binding protein (CREB) transcription factor. TATA—TATA box. TS—transcription start site.

FIG. 3 is a second schematic of a rAAV plasmid encoding full length SARS-CoV-2 S glycoprotein and corresponding structures of the S1 domain and S2 domain polypeptides in a different orientation then FIG. 2. Inset box includes the same schematic and descriptions as in FIG. 2.

FIG. 4 is a third schematic of a rAAV plasmid encoding full length SARS-CoV-2 S glycoprotein and corresponding structures of the S1 domain and S2 domain polypeptides. Inset box includes the same schematic and descriptions as in FIG. 2.

FIG. 5 is a fourth schematic of a rAAV plasmid encoding full length SARS-CoV-2 S glycoprotein.

FIG. 6 is a schematic showing cloning strategy for inserting a sequence encoding full length SARS-CoV-2 S glycoprotein into a rAAV plasmid.

### DETAILED DESCRIPTION

Provided herein are methods and materials for making and using AAVs and vectors to generate immunity to a coronavirus in a subject.

In some embodiments, the adeno-associated virus (AAV) vector including a sequence encoding a SARS-CoV-2 polypeptide or a fragment thereof. In some embodiments, the SARS-CoV-2 polypeptide is a SARS-CoV-2 S glycoprotein polypeptide or a fragment thereof. In some embodiments, the S glycoprotein or the fragment thereof includes, consists essentially, or consists of SEQ ID NO: 1. In some embodiments, the S glycoprotein or fragment thereof has an amino acid sequence that is at least 80% (e.g., at least 85%, at least 90%, at least 95%, at least 99% and at least 100%) identical to SEQ ID NO: 1.

Exemplary S glycoprotein sequence
SEQ ID NO: 1
MFVFLVLLPLVSSQCVNLTTRTQLPPAYTNSFTRGVYYPDKVFRSSVLH
STQDLFLPFFSNVTWFHAIHVSGTNGTKRFDNPVLPFNDGVYFASTEKS
NIIRGWIFGTTLDSKTQSLLIVNNATNVVIKVCEFQFCNDPFLGVYYHK
NNKSWMESEFRVYSSANNCTFEYVSQPFLMDLEGKQGNFKNLREFVFKN
IDGYFKIYSKHTPINLVRDLPQGFSALEPLVDLPIGINITRFQTLLALH
RSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVFN
ATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNL
DSKVGGNYNYLYRLFRKSNLKPFERDISTEIYQAGSTPCNGVEGFNCYF
PLQSYGFQPTNGVGYQPYRVVVLSFELLHAPATVCGPKKSTNLVKNKCV
NFNFNGLTGTGVLTESNKKFLPFQQFGRDIADTTDAVRDPQTLEILDIT
PCSFGGVSVITPGTNTSNQVAVLYQDVNCTEVPVAIHADQLTPTWRVYS
TGSNVFQTRAGCLIGAEHVNNSYECDIPIGAGICASYQTQTNSPRRARS
VASQSIIAYTMSLGAENSVAYSNNSIAIPTNFTISVTTEILPVSMTKTS
VDCTMYICGDSTECSNLLLQYGSFCTQLNRALTGIAVEQDKNTQEVFAQ
VKQIYKTPPIKDFGGFNFSQILPDPSKPSKRSFIEDLLFNKVTLADAGF
IKQYGDCLGDIAARDLICAQKFNGLTVLPPLLTDEMIAQYTSALLAGTI
TSGWTFGAGAALQIPFAMQMAYRFNGIGVTQNVLYENQKLIANQFNSAI
GKIQDSLSSTASALGKLQDVVNQNAQALNTLVKQLSSNFGAISSVLNDI
LSRLDKVEAEVQIDRLITGRLQSLQTYVTQQLIRAAEIRASANLAATKM
SECVLGQSKRVDFCGKGYHLMSFPQSAPHGVVFLHVTYVPAQEKNFTTA
PAICHDGKAHFPREGVFVSNGTHWFVTQRNFYEPQIITTDNTFVSGNCD
VVIGIVNNTVYDPLQPELDSFKEELDKYFKNHTSPDVDLGDISGINASV
VNIQKEIDRLNEVAKNLNESLIDLQELGKYEQYIKWPWYIWLGFIAGLI
AIVMVTIMLCCMTSCCSCLKGCCSCGSCCKFDEDDSEPVLKGVKLHYT

In some embodiments, the S glycoprotein polypeptide or the fragment thereof includes a S1 domain or a fragment thereof. In some embodiments, the S glycoprotein polypeptide or the fragment thereof includes, consists essentially, or consists of a S2 domain or a fragment thereof. In some embodiments, the S2 domain or the fragment thereof includes, consists essentially, or consists of SEQ ID NO: 2 or SEQ ID NO: 3. In some embodiments, the S2 domain or fragment thereof has an amino acid sequence that is at least 80% (e.g., at least 85%, at least 90%, at least 95%, at least 99% and at least 100%) identical to SEQ ID NO: 2 or SEQ ID NO: 3. In some embodiments, a fragment of the S2 domain comprises, consists, or consists essentially of amino acid number 1 to about amino acid number 580, amino acid number 1 to about amino acid number 560, amino acid number 1 to about amino acid number 540, amino acid number 1 to about amino acid number 520, amino acid number 1 to about amino acid number 500, amino acid number 1 to about amino acid number 480, amino acid number 1 to about amino acid number 460, amino acid number 1 to about amino acid number 440, amino acid number 1 to about amino acid number 420, amino acid number 1 to about amino acid number 400, amino acid number 1 to about amino acid number 380, amino acid number 1 to about amino acid number 360, amino acid number 1 to about amino acid number 340, amino acid number 1 to about amino acid number 320, amino acid number 1 to about amino acid number 300, amino acid number 1 to about amino acid number 280, amino acid number 1 to about amino acid number 260, amino acid number 1 to about amino acid number 240, amino acid number 1 to about amino acid number 220, amino acid number 1 to about amino acid number 200, amino acid number 1 to about amino acid number 180, amino acid number 1 to about amino acid number 160, amino acid number 1 to about amino acid number 140, amino acid number 1 to about amino acid number 120, amino acid number 1 to about amino acid number 100, amino acid number 1 to about amino acid number 80, amino acid number 1 to about amino acid number 60, amino acid number 1 to about amino acid number 40, amino acid number 1 to about amino acid number 20, about amino acid number 20 to about amino acid number 588, about amino acid number 20 to about amino acid number 580, about amino acid number 20 to about amino acid number 560, about amino acid number 20 to about amino acid number 540, about amino acid number 20 to about amino acid number 520, about amino acid number 20 to about amino acid number 500, about amino acid number 20 to about amino acid number 480, about amino acid number 20 to about amino acid number 460, about amino acid number 20 to about amino acid number 440, about amino acid number 20 to about amino acid number 420, about amino acid number 20 to about amino acid number 400, about amino acid number 20 to about amino acid number 380, about amino acid number 20 to about amino acid number 360, about amino acid number 20 to about amino acid number 340, about amino acid number 20 to about amino acid number 320, about amino acid number 20 to about amino acid number 300, about amino acid number 20 to about amino acid number 280, about amino acid number 20 to about amino acid number 260, about amino acid number 20 to about amino acid number 240, about amino acid number 20 to about amino acid number 220, about amino acid number 20 to about amino acid number 200, about amino acid number 20 to about amino acid number 180, about amino acid number 20 to about amino acid number 160, about amino acid number 20 to about amino acid number 140, about amino acid number 20 to about amino acid number 120, about amino acid number 20 to about amino acid number 100, about amino acid number 20 to about amino acid number 80, about amino acid number 20 to about amino acid number 60, about amino acid number 20 to about amino acid number 40, about amino acid number 40 to about amino acid number 588, about amino acid number 40 to about amino acid number 580, about amino acid number 40 to about amino acid number 560, about amino acid number 40 to about amino acid number 540, about amino acid number 40 to about amino acid number 520, about amino acid number 40 to about amino acid number 500, about amino acid number 40 to about amino acid number 480, about amino acid number 40 to about amino acid number 460, about amino acid number 40 to about amino acid number 440, about amino acid number 40 to about amino acid number 420, about amino acid number 40 to about amino acid number 400, about amino acid number 40 to about amino acid number 380, about amino acid number 40 to about amino acid number 360, about amino acid number 40 to about amino acid number 340, about amino acid number 40 to about amino acid number 320, about amino acid number 40 to about amino acid number 300, about amino acid number 40 to about amino acid number 280, about amino acid number 40 to about amino acid number 260, about amino acid number 40 to about amino acid number 240, about amino acid number 40 to about amino acid number 220, about amino acid number 40 to about amino acid number 200, about amino acid number 40 to about amino acid number 180, about amino acid number 40 to about amino acid number 160, about amino acid number 40 to about amino acid number 140, about amino acid number 40 to about amino acid number 120, about amino acid number 40 to about amino acid number 100, about amino acid number 40 to about amino acid number 80, about amino acid number 40 to about amino acid number 60, about amino acid number 60 to about amino acid number 588, about amino acid number 60 to about amino acid number 580, about amino acid number 60 to about amino acid number 560, about amino acid number 60 to about amino acid number 540, about amino acid number 60 to about amino acid number 520, about amino acid number 60 to about amino acid number 500, about amino acid number 60 to about amino acid number 480, about amino acid number 60 to about amino acid number 460, about amino acid number 60 to about amino acid number 440, about amino acid number 60 to about amino acid number 420, about amino acid number 60 to about amino acid number 400, about amino acid number 60 to about amino acid number 380, about amino acid number 60 to about amino acid number 360, about amino acid number 60 to about amino acid number 340, about amino acid number 60 to about amino acid number 320, about amino acid number 60 to about amino acid number 300, about amino acid number 60 to about amino acid number 280, about amino acid number 60 to about amino acid number 260, about amino acid number 60 to about amino acid number 240, about amino acid number 60 to about amino acid number 220, about amino acid number 60 to about amino acid number 200, about amino acid number 60 to about amino acid number 180, about amino acid number 60 to about amino acid number 160, about amino acid number 60 to about amino acid number 140, about amino acid number 60 to about amino acid number 120, about amino acid number 60 to about amino acid number 100, about amino acid number 60 to about amino acid number 80, about amino acid number 80 to about amino acid number 588, about amino acid number 80 to about amino acid number 580, about amino acid number 80 to about amino acid number 560, about amino acid number 80 to about amino acid number 540, about amino acid number 80 to about amino acid number 520, about amino acid number 80 to about amino acid number 500, about amino acid number 80 to about amino acid number 480, about amino acid number 80 to about amino acid number 460, about amino acid number 80 to about amino acid number 440, about amino acid number 80 to about amino acid number 420, about amino acid number 80 to about amino acid number 400, about amino acid number 80 to about amino acid number 380, about amino acid number 80 to about amino acid number 360, about amino acid number 80 to about amino acid number 340, about amino acid number 80 to about amino acid number 320, about amino acid number 80 to about amino acid number 300, about amino acid number 80 to about amino acid number 280, about amino acid number 80 to about amino acid number 260, about amino acid number 80 to about amino acid number 240, about amino acid number 80 to about amino acid number 220, about amino acid number 80 to about amino acid number 200, about amino acid number 80 to about amino acid number 180, about amino acid number 80 to about amino acid number 160, about amino acid number 80 to about amino acid number 140, about amino acid number 80 to about amino acid number 120, about amino acid number 80 to about amino acid number 100, about amino acid number 100 to about amino acid number 588, about amino acid number 100 to about amino acid number 580, about amino acid number 100 to about amino acid number 560, about amino acid number 100 to about amino acid number 540, about amino acid number 100 to about amino acid number 520, about amino acid number 100 to about amino acid number 500, about amino acid number 100 to about amino acid number 480, about amino acid number 100 to about amino acid number 460, about amino acid number 100 to about amino acid number 440, about amino acid number 100 to about amino acid number 420, about amino acid number 100 to about amino acid number 400, about amino acid number 100 to about amino acid number 380, about amino acid number 100 to about amino acid number 360, about amino acid number 100 to about amino acid number 340, about amino acid number 100 to about amino acid number 320, about amino acid number 100 to about amino acid number 300, about amino acid number 100 to about amino acid number 280, about amino acid number 100 to about amino acid number 260, about amino acid number 100 to about amino acid number 240, about amino acid number 100 to about amino acid number 220, about amino acid number 100 to about amino acid number 200, about amino acid number 100 to about amino acid number 180, about amino acid number 100 to about amino acid number 160, about amino acid number 100 to about amino acid number 140, about amino acid number 100 to about amino acid number 120, about amino acid number 120 to about amino acid number 588, about amino acid number 120 to about amino acid number 580, about amino acid number 120 to about amino acid number 560, about amino acid number 120 to about amino acid number 540, about amino acid number 120 to about amino acid number 520, about amino acid number 120 to about amino acid number 500, about amino acid number 120 to about amino acid number 480, about amino acid number 120 to about amino acid number 460, about amino acid number 120 to about amino acid number 440, about amino acid number 120 to about amino acid number 420, about amino acid number 120 to about amino acid number 400, about amino acid number 120 to about amino acid number 380, about amino acid number 120 to about amino acid number 360, about amino acid number 120 to about amino acid number 340, about amino acid number 120 to about amino acid number 320, about amino acid number 120 to about amino acid number 300, about amino acid number 120 to about amino acid number 280, about amino acid number 120 to about amino acid number 260, about amino acid number 120 to about amino acid number 240, about amino acid number 120 to about amino acid number 220, about amino acid number 120 to about amino acid number 200, about amino acid number 120 to about amino acid number 180, about amino acid number 120 to about amino acid number 160, about amino acid number 120 to about amino acid number 140, about amino acid number 140 to about amino acid number 588, about amino acid number 140 to about amino acid number 580, about amino acid number 140 to about amino acid number 560, about amino acid number 140 to about amino acid number 540, about amino acid number 140 to about amino acid number 520, about amino acid number 140 to about amino acid number 500, about amino acid number 140 to about amino acid number 480, about amino acid number 140 to about amino acid number 460, about amino acid number 140 to about amino acid number 440, about amino acid number 140 to about amino acid number 420, about amino acid number 140 to about amino acid number 400, about amino acid number 140 to about amino acid number 380, about amino acid number 140 to about amino acid number 360, about amino acid number 140 to about amino acid number 340, about amino acid number 140 to about amino acid number 320, about amino acid number 140 to about amino acid number 300, about amino acid number 140 to about amino acid number 280, about amino acid number 140 to about amino acid number 260, about amino acid number 140 to about amino acid number 240, about amino acid number 140 to about amino acid number 220, about amino acid number 140 to about amino acid number 200, about amino acid number 140 to about amino acid number 180, about amino acid number 140 to about amino acid number 160, about amino acid number 160 to about amino acid number 588, about amino acid number 160 to about amino acid number 580, about amino acid number 160 to about amino acid number 560, about amino acid number 160 to about amino acid number 540, about amino acid number 160 to about amino acid number 520, about amino acid number 160 to about amino acid number 500, about amino acid number 160 to about amino acid number 480, about amino acid number 160 to about amino acid number 460, about amino acid number 160 to about amino acid number 440, about amino acid number 160 to about amino acid number 420, about amino acid number 160 to about amino acid number 400, about amino acid number 160 to about amino acid number 380, about amino acid number 160 to about amino acid number 360, about amino acid number 160 to about amino acid number 340, about amino acid number 160 to about amino acid number 320, about amino acid number 160 to about amino acid number 300, about amino acid number 160 to about amino acid number 280, about amino acid number 160 to about amino acid number 260, about amino acid number 160 to about amino acid number 240, about amino acid number 160 to about amino acid number 220, about amino acid number 160 to about amino acid number 200, about amino acid number 160 to about amino acid number 180, about amino acid number 180 to about amino acid number 588, about amino acid number 180 to about amino acid number 580, about amino acid number 180 to about amino acid number 560, about amino acid number 180 to about amino acid number 540, about amino acid number 180 to about amino acid number 520, about amino acid number 180 to about amino acid number 500, about amino acid number 180 to about amino acid number 480, about amino acid number 180 to about amino acid number 460, about amino acid number 180 to about amino acid number 440, about amino acid number 180 to about amino acid number 420, about amino acid number 180 to about amino acid number 400, about amino acid number 180 to about amino acid number 380, about amino acid number 180 to about amino acid number 360, about amino acid number 180 to about amino acid number 340, about amino acid number 180 to about amino acid number 320, about amino acid number 180 to about amino acid number 300, about amino acid number 180 to about amino acid number 280, about amino acid number 180 to about amino acid number 260, about amino acid number 180 to about amino acid number 240, about amino acid number 180 to about amino acid number 220, about amino acid number 180 to about amino acid number 200, about amino acid number 200 to about amino acid number 588, about amino acid number 200 to about amino acid number 580, about amino acid number 200 to about amino acid number 560, about amino acid number 200 to about amino acid number 540, about amino acid number 200 to about amino acid number 520, about amino acid number 200 to about amino acid number 500, about amino acid number 200 to about amino acid number 480, about amino acid number 200 to about amino acid number 460, about amino acid number 200 to about amino acid number 440, about amino acid number 200 to about amino acid number 420, about amino acid number 200 to about amino acid number 400, about amino acid number 200 to about amino acid number 380, about amino acid number 200 to about amino acid number 360, about amino acid number 200 to about amino acid number 340, about amino acid number 200 to about amino acid number 320, about amino acid number 200 to about amino acid number 300, about amino acid number 200 to about amino acid number 280, about amino acid number 200 to about amino acid number 260, about amino acid number 200 to about amino acid number 240, about amino acid number 200 to about amino acid number 220, about amino acid number 220 to about amino acid number 588, about amino acid number 220 to about amino acid number 580, about amino acid number 220 to about amino acid number 560, about amino acid number 220 to about amino acid number 540, about amino acid number 220 to about amino acid number 520, about amino acid number 220 to about amino acid number 500, about amino acid number 220 to about amino acid number 480, about amino acid number 220 to about amino acid number 460, about amino acid number 220 to about amino acid number 440, about amino acid number 220 to about amino acid number 420, about amino acid number 220 to about amino acid number 400, about amino acid number 220 to about amino acid number 380, about amino acid number 220 to about amino acid number 360, about amino acid number 220 to about amino acid number 340, about amino acid number 220 to about amino acid number 320, about amino acid number 220 to about amino acid number 300, about amino acid number 220 to about amino acid number 280, about amino acid number 220 to about amino acid number 260, about amino acid number 220 to about amino acid number 240, about amino acid number 240 to about amino acid number 588, about amino acid number 240 to about amino acid number 580, about amino acid number 240 to about amino acid number 560, about amino acid number 240 to about amino acid number 540, about amino acid number 240 to about amino acid number 520, about amino acid number 240 to about amino acid number 500, about amino acid number 240 to about amino acid number 480, about amino acid number 240 to about amino acid number 460, about amino acid number 240 to about amino acid number 440, about amino acid number 240 to about amino acid number 420, about amino acid number 240 to about amino acid number 400, about amino acid number 240 to about amino acid number 380, about amino acid number 240 to about amino acid number 360, about amino acid number 240 to about amino acid number 340, about amino acid number 240 to about amino acid number 320, about amino acid number 240 to about amino acid number 300, about amino acid number 240 to about amino acid number 280, about amino acid number 240 to about amino acid number 260, about amino acid number 260 to about amino acid number 588, about amino acid number 260 to about amino acid number 580, about amino acid number 260 to about amino acid number 560, about amino acid number 260 to about amino acid number 540, about amino acid number 260 to about amino acid number 520, about amino acid number 260 to about amino acid number 500, about amino acid number 260 to about amino acid number 480, about amino acid number 260 to about amino acid number 460, about amino acid number 260 to about amino acid number 440, about amino acid number 260 to about amino acid number 420, about amino acid number 260 to about amino acid number 400, about amino acid number 260 to about amino acid number 380, about amino acid number 260 to about amino acid number 360, about amino acid number 260 to about amino acid number 340, about amino acid number 260 to about amino acid number 320, about amino acid number 260 to about amino acid number 300, about amino acid number 260 to about amino acid number 280, about amino acid number 280 to about amino acid number 588, about amino acid number 280 to about amino acid number 580, about amino acid number 280 to about amino acid number 560, about amino acid number 280 to about amino acid number 540, about amino acid number 280 to about amino acid number 520, about amino acid number 280 to about amino acid number 500, about amino acid number 280 to about amino acid number 480, about amino acid number 280 to about amino acid number 460, about amino acid number 280 to about amino acid number 440, about amino acid number 280 to about amino acid number 420, about amino acid number 280 to about amino acid number 400, about amino acid number 280 to about amino acid number 380, about amino acid number 280 to about amino acid number 360, about amino acid number 280 to about amino acid number 340, about amino acid number 280 to about amino acid number 320, about amino acid number 280 to about amino acid number 300, about amino acid number 300 to about amino acid number 588, about amino acid number 300 to about amino acid number 580, about amino acid number 300 to about amino acid number 560, about amino acid number 300 to about amino acid number 540, about amino acid number 300 to about amino acid number 520, about amino acid number 300 to about amino acid number 500, about amino acid number 300 to about amino acid number 480, about amino acid number 300 to about amino acid number 460, about amino acid number 300 to about amino acid number 440, about amino acid number 300 to about amino acid number 420, about amino acid number 300 to about amino acid number 400, about amino acid number 300 to about amino acid number 380, about amino acid number 300 to about amino acid number 360, about amino acid number 300 to about amino acid number 340, about amino acid number 300 to about amino acid number 320, about amino acid number 320 to about amino acid number 588, about amino acid number 320 to about amino acid number 580, about amino acid number 320 to about amino acid number 560, about amino acid number 320 to about amino acid number 540, about amino acid number 320 to about amino acid number 520, about amino acid number 320 to about amino acid number 500, about amino acid number 320 to about amino acid number 480, about amino acid number 320 to about amino acid number 460, about amino acid number 320 to about amino acid number 440, about amino acid number 320 to about amino acid number 420, about amino acid number 320 to about amino acid number 400, about amino acid number 320 to about amino acid number 380, about amino acid number 320 to about amino acid number 360, about amino acid number 320 to about amino acid number 340, about amino acid number 340 to about amino acid number 588, about amino acid number 340 to about amino acid number 580, about amino acid number 340 to about amino acid number 560, about amino acid number 340 to about amino acid number 540, about amino acid number 340 to about amino acid number 520, about amino acid number 340 to about amino acid number 500, about amino acid number 340 to about amino acid number 480, about amino acid number 340 to about amino acid number 460, about amino acid number 340 to about amino acid number 440, about amino acid number 340 to about amino acid number 420, about amino acid number 340 to about amino acid number 400, about amino acid number 340 to about amino acid number 380, about amino acid number 340 to about amino acid number 360, about amino acid number 360 to about amino acid number 588, about amino acid number 360 to about amino acid number 580, about amino acid number 360 to about amino acid number 560, about amino acid number 360 to about amino acid number 540, about amino acid number 360 to about amino acid number 520, about amino acid number 360 to about amino acid number 500, about amino acid number 360 to about amino acid number 480, about amino acid number 360 to about amino acid number 460, about amino acid number 360 to about amino acid number 440, about amino acid number 360 to about amino acid number 420, about amino acid number 360 to about amino acid number 400, about amino acid number 360 to about amino acid number 380, about amino acid number 380 to about amino acid number 588, about amino acid number 380 to about amino acid number 580, about amino acid number 380 to about amino acid number 560, about amino acid number 380 to about amino acid number 540, about amino acid number 380 to about amino acid number 520, about amino acid number 380 to about amino acid number 500, about amino acid number 380 to about amino acid number 480, about amino acid number 380 to about amino acid number 460, about amino acid number 380 to about amino acid number 440, about amino acid number 380 to about amino acid number 420, about amino acid number 380 to about amino acid number 400, about amino acid number 400 to about amino acid number 588, about amino acid number 400 to about amino acid number 580, about amino acid number 400 to about amino acid number 560, about amino acid number 400 to about amino acid number 540, about amino acid number 400 to about amino acid number 520, about amino acid number 400 to about amino acid number 500, about amino acid number 400 to about amino acid number 480, about amino acid number 400 to about amino acid number 460, about amino acid number 400 to about amino acid number 440, about amino acid number 400 to about amino acid number 420, about amino acid number 420 to about amino acid number 588, about amino acid number 420 to about amino acid number 580, about amino acid number 420 to about amino acid number 560, about amino acid number 420 to about amino acid number 540, about amino acid number 420 to about amino acid number 520, about amino acid number 420 to about amino acid number 500, about amino acid number 420 to about amino acid number 480, about amino acid number 420 to about amino acid number 460, about amino acid number 420 to about amino acid number 440, about amino acid number 440 to about amino acid number 588, about amino acid number 440 to about amino acid number 580, about amino acid number 440 to about amino acid number 560, about amino acid number 440 to about amino acid number 540, about amino acid number 440 to about amino acid number 520, about amino acid number 440 to about amino acid number 500, about amino acid number 440 to about amino acid number 480, about amino acid number 440 to about amino acid number 460, about amino acid number 460 to about amino acid number 588, about amino acid number 460 to about amino acid number 580, about amino acid number 460 to about amino acid number 560, about amino acid number 460 to about amino acid number 540, about amino acid number 460 to about amino acid number 520, about amino acid number 460 to about amino acid number 500, about amino acid number 460 to about amino acid number 480, about amino acid number 480 to about amino acid number 588, about amino acid number 480 to about amino acid number 580, about amino acid number 480 to about amino acid number 560, about amino acid number 480 to about amino acid number 540, about amino acid number 480 to about amino acid number 520, about amino acid number 480 to about amino acid number 500, about amino acid number 500 to about amino acid number 588, about amino acid number 500 to about amino acid number 580, about amino acid number 500 to about amino acid number 560, about amino acid number 500 to about amino acid number 540, about amino acid number 500 to about amino acid number 520, about amino acid number 520 to about amino acid number 588, about amino acid number 520 to about amino acid number 580, about amino acid number 520 to about amino acid number 560, about amino acid number 520 to about amino acid number 540, about amino acid number 540 to about amino acid number 588, about amino acid number 540 to about amino acid number 580, about amino acid number 540 to about amino acid number 560, about amino acid number 560 to about amino acid number 588, or about amino acid number 560 to about amino acid number 580, of SEQ ID NO: 2.

In some embodiments, a fragment of the S2 domain comprises, consists, or consists essentially of amino acid number 1 to about amino acid number 458, amino acid number 1 to about amino acid number 440, amino acid number 1 to about amino acid number 420, amino acid number 1 to about amino acid number 400, amino acid number 1 to about amino acid number 380, amino acid number 1 to about amino acid number 360, amino acid number 1 to about amino acid number 340, amino acid number 1 to about amino acid number 320, amino acid number 1 to about amino acid number 300, amino acid number 1 to about amino acid number 280, amino acid number 1 to about amino acid number 260, amino acid number 1 to about amino acid number 240, amino acid number 1 to about amino acid number 220, amino acid number 1 to about amino acid number 200, amino acid number 1 to about amino acid number 180, amino acid number 1 to about amino acid number 160, amino acid number 1 to about amino acid number 140, amino acid number 1 to about amino acid number 120, amino acid number 1 to about amino acid number 100, amino acid number 1 to about amino acid number 80, amino acid number 1 to about amino acid number 60, amino acid number 1 to about amino acid number 40, amino acid number 1 to about amino acid number 20, about amino acid number 20 to about amino acid number 458, about amino acid number 20 to about amino acid number 440, about amino acid number 20 to about amino acid number 420, about amino acid number 20 to about amino acid number 400, about amino acid number 20 to about amino acid number 380, about amino acid number 20 to about amino acid number 360, about amino acid number 20 to about amino acid number 340, about amino acid number 20 to about amino acid number 320, about amino acid number 20 to about amino acid number 300, about amino acid number 20 to about amino acid number 280, about amino acid number 20 to about amino acid number 260, about amino acid number 20 to about amino acid number 240, about amino acid number 20 to about amino acid number 220, about amino acid number 20 to about amino acid number 200, about amino acid number 20 to about amino acid number 180, about amino acid number 20 to about amino acid number 160, about amino acid number 20 to about amino acid number 140, about amino acid number 20 to about amino acid number 120, about amino acid number 20 to about amino acid number 100, about amino acid number 20 to about amino acid number 80, about amino acid number 20 to about amino acid number 60, about amino acid number 20 to about amino acid number 40, about amino acid number 40 to about amino acid number 458, about amino acid number 40 to about amino acid number 440, about amino acid number 40 to about amino acid number 420, about amino acid number 40 to about amino acid number 400, about amino acid number 40 to about amino acid number 380, about amino acid number 40 to about amino acid number 360, about amino acid number 40 to about amino acid number 340, about amino acid number 40 to about amino acid number 320, about amino acid number 40 to about amino acid number 300, about amino acid number 40 to about amino acid number 280, about amino acid number 40 to about amino acid number 260, about amino acid number 40 to about amino acid number 240, about amino acid number 40 to about amino acid number 220, about amino acid number 40 to about amino acid number 200, about amino acid number 40 to about amino acid number 180, about amino acid number 40 to about amino acid number 160, about amino acid number 40 to about amino acid number 140, about amino acid number 40 to about amino acid number 120, about amino acid number 40 to about amino acid number 100, about amino acid number 40 to about amino acid number 80, about amino acid number 40 to about amino acid number 60, about amino acid number 60 to about amino acid number 458, about amino acid number 60 to about amino acid number 440, about amino acid number 60 to about amino acid number 420, about amino acid number 60 to about amino acid number 400, about amino acid number 60 to about amino acid number 380, about amino acid number 60 to about amino acid number 360, about amino acid number 60 to about amino acid number 340, about amino acid number 60 to about amino acid number 320, about amino acid number 60 to about amino acid number 300, about amino acid number 60 to about amino acid number 280, about amino acid number 60 to about amino acid number 260, about amino acid number 60 to about amino acid number 240, about amino acid number 60 to about amino acid number 220, about amino acid number 60 to about amino acid number 200, about amino acid number 60 to about amino acid number 180, about amino acid number 60 to about amino acid number 160, about amino acid number 60 to about amino acid number 140, about amino acid number 60 to about amino acid number 120, about amino acid number 60 to about amino acid number 100, about amino acid number 60 to about amino acid number 80, about amino acid number 80 to about amino acid number 458, about amino acid number 80 to about amino acid number 440, about amino acid number 80 to about amino acid number 420, about amino acid number 80 to about amino acid number 400, about amino acid number 80 to about amino acid number 380, about amino acid number 80 to about amino acid number 360, about amino acid number 80 to about amino acid number 340, about amino acid number 80 to about amino acid number 320, about amino acid number 80 to about amino acid number 300, about amino acid number 80 to about amino acid number 280, about amino acid number 80 to about amino acid number 260, about amino acid number 80 to about amino acid number 240, about amino acid number 80 to about amino acid number 220, about amino acid number 80 to about amino acid number 200, about amino acid number 80 to about amino acid number 180, about amino acid number 80 to about amino acid number 160, about amino acid number 80 to about amino acid number 140, about amino acid number 80 to about amino acid number 120, about amino acid number 80 to about amino acid number 100, about amino acid number 100 to about amino acid number 458, about amino acid number 100 to about amino acid number 440, about amino acid number 100 to about amino acid number 420, about amino acid number 100 to about amino acid number 400, about amino acid number 100 to about amino acid number 380, about amino acid number 100 to about amino acid number 360, about amino acid number 100 to about amino acid number 340, about amino acid number 100 to about amino acid number 320, about amino acid number 100 to about amino acid number 300, about amino acid number 100 to about amino acid number 280, about amino acid number 100 to about amino acid number 260, about amino acid number 100 to about amino acid number 240, about amino acid number 100 to about amino acid number 220, about amino acid number 100 to about amino acid number 200, about amino acid number 100 to about amino acid number 180, about amino acid number 100 to about amino acid number 160, about amino acid number 100 to about amino acid number 140, about amino acid number 100 to about amino acid number 120, about amino acid number 120 to about amino acid number 458, about amino acid number 120 to about amino acid number 440, about amino acid number 120 to about amino acid number 420, about amino acid number 120 to about amino acid number 400, about amino acid number 120 to about amino acid number 380, about amino acid number 120 to about amino acid number 360, about amino acid number 120 to about amino acid number 340, about amino acid number 120 to about amino acid number 320, about amino acid number 120 to about amino acid number 300, about amino acid number 120 to about amino acid number 280, about amino acid number 120 to about amino acid number 260, about amino acid number 120 to about amino acid number 240, about amino acid number 120 to about amino acid number 220, about amino acid number 120 to about amino acid number 200, about amino acid number 120 to about amino acid number 180, about amino acid number 120 to about amino acid number 160, about amino acid number 120 to about amino acid number 140, about amino acid number 140 to about amino acid number 458, about amino acid number 140 to about amino acid number 440, about amino acid number 140 to about amino acid number 420, about amino acid number 140 to about amino acid number 400, about amino acid number 140 to about amino acid number 380, about amino acid number 140 to about amino acid number 360, about amino acid number 140 to about amino acid number 340, about amino acid number 140 to about amino acid number 320, about amino acid number 140 to about amino acid number 300, about amino acid number 140 to about amino acid number 280, about amino acid number 140 to about amino acid number 260, about amino acid number 140 to about amino acid number 240, about amino acid number 140 to about amino acid number 220, about amino acid number 140 to about amino acid number 200, about amino acid number 140 to about amino acid number 180, about amino acid number 140 to about amino acid number 160, about amino acid number 160 to about amino acid number 458, about amino acid number 160 to about amino acid number 440, about amino acid number 160 to about amino acid number 420, about amino acid number 160 to about amino acid number 400, about amino acid number 160 to about amino acid number 380, about amino acid number 160 to about amino acid number 360, about amino acid number 160 to about amino acid number 340, about amino acid number 160 to about amino acid number 320, about amino acid number 160 to about amino acid number 300, about amino acid number 160 to about amino acid number 280, about amino acid number 160 to about amino acid number 260, about amino acid number 160 to about amino acid number 240, about amino acid number 160 to about amino acid number 220, about amino acid number 160 to about amino acid number 200, about amino acid number 160 to about amino acid number 180, about amino acid number 180 to about amino acid number 458, about amino acid number 180 to about amino acid number 440, about amino acid number 180 to about amino acid number 420, about amino acid number 180 to about amino acid number 400, about amino acid number 180 to about amino acid number 380, about amino acid number 180 to about amino acid number 360, about amino acid number 180 to about amino acid number 340, about amino acid number 180 to about amino acid number 320, about amino acid number 180 to about amino acid number 300, about amino acid number 180 to about amino acid number 280, about amino acid number 180 to about amino acid number 260, about amino acid number 180 to about amino acid number 240, about amino acid number 180 to about amino acid number 220, about amino acid number 180 to about amino acid number 200, about amino acid number 200 to about amino acid number 458, about amino acid number 200 to about amino acid number 440, about amino acid number 200 to about amino acid number 420, about amino acid number 200 to about amino acid number 400, about amino acid number 200 to about amino acid number 380, about amino acid number 200 to about amino acid number 360, about amino acid number 200 to about amino acid number 340, about amino acid number 200 to about amino acid number 320, about amino acid number 200 to about amino acid number 300, about amino acid number 200 to about amino acid number 280, about amino acid number 200 to about amino acid number 260, about amino acid number 200 to about amino acid number 240, about amino acid number 200 to about amino acid number 220, about amino acid number 220 to about amino acid number 458, about amino acid number 220 to about amino acid number 440, about amino acid number 220 to about amino acid number 420, about amino acid number 220 to about amino acid number 400, about amino acid number 220 to about amino acid number 380, about amino acid number 220 to about amino acid number 360, about amino acid number 220 to about amino acid number 340, about amino acid number 220 to about amino acid number 320, about amino acid number 220 to about amino acid number 300, about amino acid number 220 to about amino acid number 280, about amino acid number 220 to about amino acid number 260, about amino acid number 220 to about amino acid number 240, about amino acid number 240 to about amino acid number 458, about amino acid number 240 to about amino acid number 440, about amino acid number 240 to about amino acid number 420, about amino acid number 240 to about amino acid number 400, about amino acid number 240 to about amino acid number 380, about amino acid number 240 to about amino acid number 360, about amino acid number 240 to about amino acid number 340, about amino acid number 240 to about amino acid number 320, about amino acid number 240 to about amino acid number 300, about amino acid number 240 to about amino acid number 280, about amino acid number 240 to about amino acid number 260, about amino acid number 260 to about amino acid number 458, about amino acid number 260 to about amino acid number 440, about amino acid number 260 to about amino acid number 420, about amino acid number 260 to about amino acid number 400, about amino acid number 260 to about amino acid number 380, about amino acid number 260 to about amino acid number 360, about amino acid number 260 to about amino acid number 340, about amino acid number 260 to about amino acid number 320, about amino acid number 260 to about amino acid number 300, about amino acid number 260 to about amino acid number 280, about amino acid number 280 to about amino acid number 458, about amino acid number 280 to about amino acid number 440, about amino acid number 280 to about amino acid number 420, about amino acid number 280 to about amino acid number 400, about amino acid number 280 to about amino acid number 380, about amino acid number 280 to about amino acid number 360, about amino acid number 280 to about amino acid number 340, about amino acid number 280 to about amino acid number 320, about amino acid number 280 to about amino acid number 300, about amino acid number 300 to about amino acid number 458, about amino acid number 300 to about amino acid number 440, about amino acid number 300 to about amino acid number 420, about amino acid number 300 to about amino acid number 400, about amino acid number 300 to about amino acid number 380, about amino acid number 300 to about amino acid number 360, about amino acid number 300 to about amino acid number 340, about amino acid number 300 to about amino acid number 320, about amino acid number 320 to about amino acid number 458, about amino acid number 320 to about amino acid number 440, about amino acid number 320 to about amino acid number 420, about amino acid number 320 to about amino acid number 400, about amino acid number 320 to about amino acid number 380, about amino acid number 320 to about amino acid number 360, about amino acid number 320 to about amino acid number 340, about amino acid number 340 to about amino acid number 458, about amino acid number 340 to about amino acid number 440, about amino acid number 340 to about amino acid number 420, about amino acid number 340 to about amino acid number 400, about amino acid number 340 to about amino acid number 380, about amino acid number 340 to about amino acid number 360, about amino acid number 360 to about amino acid number 458, about amino acid number 360 to about amino acid number 440, about amino acid number 360 to about amino acid number 420, about amino acid number 360 to about amino acid number 400, about amino acid number 360 to about amino acid number 380, about amino acid number 380 to about amino acid number 458, about amino acid number 380 to about amino acid number 440, about amino acid number 380 to about amino acid number 420, about amino acid number 380 to about amino acid number 400, about amino acid number 400 to about amino acid number 458, about amino acid number 400 to about amino acid number 440, about amino acid number 400 to about amino acid number 420, about amino acid number 420 to about amino acid number 458, about amino acid number 420 to about amino acid number 440, about amino acid number 440 to about amino acid number 458, of SEQ ID NO: 3.

Exemplary S2 domain sequence
SEQ ID NO: 2
SVASQSIIAYTMSLGAENSVAYSNNSIAIPTNFTISVTTEILPVSMTKT
SVDCTMYICGDSTECSNLLLQYGSFCTQLNRALTGIAVEQDKNTQEVFA
QVKQIYKTPPIKDFGGFNFSQILPDPSKPSKRSFIEDLLFNKVTLADAG
FIKQYGDCLGDIAARDLICAQKFNGLTVLPPLLTDEMIAQYTSALLAGT
ITSGWTFGAGAALQIPFAMQMAYRFNGIGVTQNVLYENQKLIANQFNSA
IGKIQDSLSSTASALGKLQDVVNQNAQALNTLVKQLSSNFGAISSVLND
ILSRLDKVEAEVQIDRLITGRLQSLQTYVTQQLIRAAEIRASANLAATK
MSECVLGQSKRVDFCGKGYHLMSFPQSAPHGVVFLHVTYVPAQEKNFTT
APAICHDGKAHFPREGVFVSNGTHWFVTQRNFYEPQIITTDNTFVSGNC
DVVIGIVNNTVYDPLQPELDSFKEELDKYFKNHTSPDVDLGDISGINAS
VVNIQKEIDRLNEVAKNLNESLIDLQELGKYEQYIKWPWYIWLGFIAGL
IAIVMVTIMLCCMTSCCSCLKGCCSCGSCCKFDEDDSEPVLKGVKLHYT
Exemplary S2 domain sequence
SEQ ID NO: 3
SFIEDLLFNKVTLADAGFIKQYGDCLGDIAARDLICAQKFNGLTVLPPL
LTDEMIAQYTSALLAGTITSGWTFGAGAALQIPFAMQMAYRFNGIGVTQ
NVLYENQKLIANQFNSAIGKIQDSLSSTASALGKLQDVVNQNAQALNTL
VKQLSSNFGAISSVLNDILSRLDKVEAEVQIDRLITGRLQSLQTYVTQQ
LIRAAEIRASANLAATKMSECVLGQSKRVDFCGKGYHLMSFPQSAPHGV
VFLHVTYVPAQEKNFTTAPAICHDGKAHFPREGVFVSNGTHWFVTQRNF
YEPQIITTDNTFVSGNCDVVIGIVNNTVYDPLQPELDSFKEELDKYFKN
HTSPDVDLGDISGINASVVNIQKEIDRLNEVAKNLNESLIDLQELGKYE
QYIKWPWYIWLGFIAGLIAIVMVTIMLCCMTSCCSCLKGCCSCGSCCKF
DEDDSEPVLKGVKLHYT

In some embodiments, the AAV also includes a sequence encoding a transmembrane domain, wherein the sequence encoding the transmembrane domain is operably linked to the sequence encoding the S glycoprotein polypeptide or the fragment thereof. In some embodiments, the transmembrane domain includes a sequence of SEQ ID NO: 4, or a fragment thereof. In some embodiments, the transmembrane domain or fragment thereof includes an amino acid sequence that is at least 80% (e.g., at least 85%, at least 90%, at least 95%, at least 99% and at least 100%) identical to SEQ ID NO: 4.

Exemplary transmembrane domain sequence
SEQ ID NO: 4
WPWYIWLGFIAGLIAIVMVTIML

In some embodiments, the SARS-CoV-2 polypeptide or the fragment thereof is a nucleocapsid protein or a fragment thereof. In some embodiments, the nucleocapsid polypeptide or the fragment thereof includes a sequence of SEQ ID NO: 5 or a fragment thereof. In some embodiments, the nucleocapsid polypeptide or a fragment thereof includes an amino acid sequence that is at least 80% (e.g., at least 85%, at least 90%, at least 95%, at least 99% and at least 100%) identical to SEQ ID NO: 5.

Exemplary nucleocapsid polypeptide sequence
SEQ ID NO: 5
MSDNGPQNQRNAPRITFGGPSDSTGSNQNGERSGARSKQRRPQGLPNNT
ASWFTALTQHGKEDLKFPRGQGVPINTNSSPDDQIGYYRRATRRIRGGD
GKMKDLSPRWYFYYLGTGPEAGLPYGANKDGIIWVATEGALNTPKDHIG
TRNPANNAAIVLQLPQGTTLPKGFYAEGSRGGSQASSRSSSRSRNSSRN
STPGSSRGTSPARMAGNGGDAALALLLLDRLNQLESKMSGKGQQQQGQT
VTKKSAAEASKKPRQKRTATKAYNVTQAFGRRGPEQTQGNFGDQELIRQ
GTDYKHWPQIAQFAPSASAFFGMSRIGMEVTPSGTWLTYTGAIKLDDKD
PNFKDQVILLNKHIDAYKTFPPTEPKKDKKKKADETQALPQRQKKQQTV
TLLPAADLDDFSKQLQQSMSSADSTQA

In some embodiments, the SARS-CoV-2 polypeptide or the fragment thereof is an envelope polypeptide or a fragment thereof.

In some embodiments, the SARS-CoV-2 polypeptide or the fragment thereof is a membrane polypeptide or a fragment thereof.

In some embodiments, the AAV also includes a sequence encoding a signal sequence, wherein the sequence encoding the signal sequence is operatively linked to the sequence encoding the SARS CoV-2 polypeptide or the fragment thereof. In some embodiments, the signal sequence includes a sequence of SEQ ID NO: 6. In some embodiments, the signal sequence includes an amino acid sequence that is at least 80% (e.g., at least 85%, at least 90%, at least 95%, at least 99% and at least 100%) identical to SEQ ID NO: 6.

Exemplary signal sequence
SEQ ID NO: 6
MFVFLVLLPLVSSQC

In some embodiments, the AAV also includes a promoter operatively linked to the sequence encoding the SARS CoV-2 polypeptide or the fragment thereof. In some embodiments, the promoter is a muscle-specific promoter. In some embodiments, the muscle-specific promoter is selected from the group consisting of: skeletal β-actin, myosin light chain 2A, dystrophin, SPc-512, muscle creatine kinase, SPc-512, and synthetic muscle promoters. In some embodiments, the promoter is selected from the group consisting of: CMV, RSV, SV40, β-actin, PGK, and EF1 promoters.

In some embodiments, the AAV vector includes an AAV serotype 6 (AAV6) capsid protein. In some embodiments, the AAV vector includes an AAV serotype 9 (AAV9) capsid protein. In some embodiments, the AAV vector includes an Anc80, Anc80Lib, Anc 81, Anc82, Anc83, Anc84, Anc110, Anc113, Anc126, Anc127 or another Ancestral AAV capsid protein.

Also provided herein are pharmaceutical composition including any of the AAV or AAV vectors described herein. In some embodiments, the pharmaceutical composition also includes an adjuvant. In some embodiments, the adjuvant is a CpG adjuvant.

Also provided herein are vaccine composition including any of the AAV or AAV vectors described herein. In some embodiments, the vaccine composition also includes an adjuvant. In some embodiments, the adjuvant is a CpG adjuvant.

In another aspect, this document provides a vector including a sequence encoding a SARS-CoV-2 polypeptide or a fragment thereof. In some embodiments, the SARS-CoV-2 polypeptide is a SARS-CoV-2 S glycoprotein polypeptide or a fragment thereof. In some embodiments, the S glycoprotein polypeptide or the fragment thereof includes a S1 domain or a fragment thereof. In some embodiments, the S glycoprotein polypeptide or the fragment thereof includes, consists essentially, or consists of a S2 domain or a fragment thereof. In some embodiments, the S2 domain or the fragment thereof includes, consists essentially, or consists of SEQ ID NO: 2 or SEQ ID NO: 3.

In some embodiments, the vector also includes a sequence encoding a transmembrane domain, wherein the sequence encoding the transmembrane domain is operably linked to the sequence encoding the S glycoprotein polypeptide or the fragment thereof. In some embodiments, the transmembrane domain includes a sequence of SEQ ID NO: 4 or a fragment thereof.

In some embodiments, the SARS-CoV-2 polypeptide or the fragment thereof is a nucleocapsid protein or a fragment thereof. In some embodiments, the nucleocapsid polypeptide or the fragment thereof includes a sequence of SEQ ID NO: 5 or a fragment thereof.

In some embodiments, the SARS-CoV-2 polypeptide or the fragment thereof is an envelope polypeptide or a fragment thereof.

In some embodiments, the SARS-CoV-2 polypeptide or the fragment thereof is a membrane polypeptide or a fragment thereof.

In some embodiments, the vector also includes a sequence encoding a signal sequence, wherein the sequence encoding the signal sequence is operatively linked to the sequence encoding the SARS CoV-2 polypeptide or the fragment thereof. In some embodiments, the signal sequence includes a sequence of SEQ ID NO: 6.

In some embodiments, the vector includes a promoter operatively linked to the sequence encoding the SARS CoV-2 polypeptide or the fragment thereof. In some embodiments, the promoter is a muscle-specific promoter. In some embodiments, the muscle-specific promoter is selected from the group consisting of: skeletal β-actin, myosin light chain 2A, dystrophin, muscle creatine kinase, SPc-512, and synthetic muscle promoters. In some embodiments, the promoter is selected from the group consisting of: CMV, RSV, SV40, β-actin, PGK, and EF1 promoters.

In some embodiments, the vector is a viral vector. In some embodiments, the viral vector is a lentivirus vector or herpes virus vector. In some embodiments, the vector is a plasmid.

Also provided herein are pharmaceutical compositions including any of the vectors described herein. In some embodiments, the pharmaceutical composition includes an adjuvant. In some embodiments, the adjuvant is a CpG adjuvant.

Also provided herein are vaccine composition including any of the vectors described herein. In some embodiments, the vaccine composition also includes an adjuvant. In some embodiments, the adjuvant is a CpG adjuvant.

In another aspect, this document provides a method of inducing at least partial immunity to a coronavirus in a subject, the method including administering to the subject a therapeutically effective amount of any of the AAV or AAV vectors described herein, any of the vectors described herein, any of the pharmaceutical composition described herein, or any of the vaccine composition described herein. In some embodiments, the administering results in at least partial immunity to the coronavirus due to humoral immunity to the coronavirus. In some embodiments, the administering results in robust T-cell mediated immunity to coronavirus. In some embodiments, the at least partial immunity to the coronavirus is humoral immunity to the coronavirus. In some embodiments, the at least partial immunity to the coronavirus is T-cell mediated immunity to the coronavirus. In some embodiments, the administering results in an increase in titer of antibodies that specifically bind to the SARS-CoV-2 polypeptide or the fragment thereof in the subject. In some embodiments, the administering results in a decrease in the rate of infection of the coronavirus in the subject. In some embodiments, the method also includes administering an adjuvant to the subject. In some embodiments, the adjuvant is a CpG adjuvant.

In some embodiments, the subject has been identified as not having previously had a coronavirus infection.

In some embodiments, prior to the administering step, the subject has been identified as not having a significant titer of antibodies that bind specifically to the SARS-CoV-2 polypeptide of the fragment thereof.

In some embodiments, the coronavirus is SARS-CoV-2 (COVID-19).

In some embodiments, the subject has been previously identified as having one or more medical conditions selected from the group consisting of: chronic lung disease, moderate asthma, severe asthma, heart conditions, diabetes, obesity, liver disease, chronic kidney disease, and a weakened or suppressed immune system. In some embodiments, the subject having a weakened or suppressed immune system is a subject receiving a cancer treatment, a smoker, a subject who is a transplant recipient, a subject having HIV or AIDS, or a subject receiving a corticosteroid or any other immunosuppressant drug. In some embodiments, the subject having a weakened or suppressed immune system is an elderly subject.

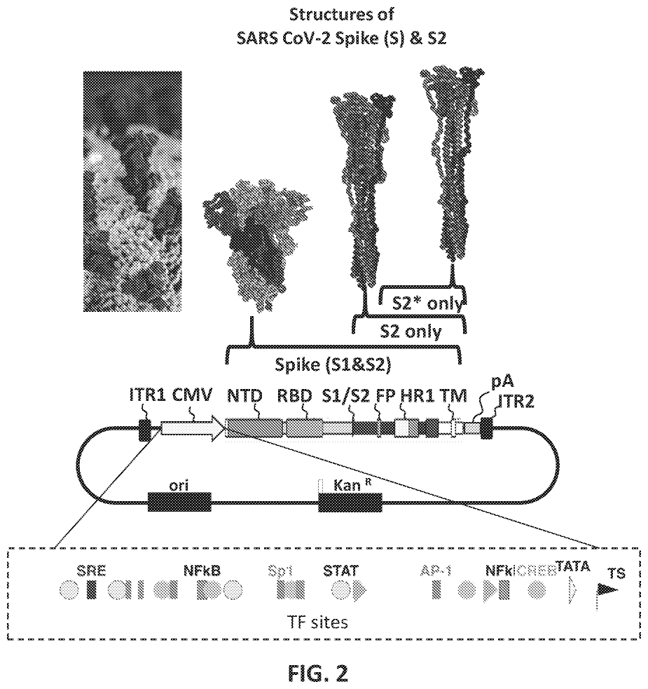

As seen in FIG. 2, the proteins in this figure are shown approximately to scale, when the S1 domain is blown out, the S2 domain refolds to create a transient structure that is present as the virus attempts to fuse to the host cell. The S2 residues are much more conserved than the S1 residues. This disclosure is based in part on the discovery that that the outermost S1 residues and their state of glycosylation creates a broad surface for binding of neutralizing and non-neutralizing antibodies, any combination of which have the capacity to produce Antibody-Dependent Enhancement (ADE) in a fraction of the hosts. The use of the S2 and S2* subfragments, with or without a signal sequence to drive protein synthesis in the ER/golgi apparatus for transfer to the cell surface, will not only minimize the risk of ADE, but also maximize the durability of the immunization because the S2 residues are much more conserved than those on the surface of S1. The stable conformation of the S2 and/or S2* residues as illustrated in FIG. 2 and the stability of S2 and/or S2* residues during the infectious process indicates that antibodies formed against these epitopes will have the opportunity to impede fusion and productive infection.

### Vectors

Also provided herein are nucleic acid sequences that encode any of the SARS-CoV-2 polypeptides described herein. For example, nucleic acid sequences are included that encode for a S glycoprotein polypeptide, a S1 domain of S glycoprotein polypeptide, a S2 domain of a S glycoprotein polypeptide, a nucleocapsid polypeptide, a membrane polypeptide, or an envelope polypeptide. Also provided herein are vectors that include any of the nucleic acid sequences encoding any of the polypeptides described herein. For example, the polypeptides include, without limitation, a S glycoprotein polypeptide, a S1 domain of S glycoprotein polypeptide, a S2 domain of a S glycoprotein polypeptide, a nucleocapsid polypeptide, a membrane polypeptide, or an envelope polypeptide.

As used herein, the term “vector” means a composition including a polynucleotide capable of carrying at least one exogenous nucleic acid fragment, e.g., a plasmid vector, a transposon, a cosmid, an artificial chromosome (e.g., a human artificial chromosome (HAC), a yeast artificial chromosome (YAC), a bacterial artificial chromosome (BAC), or a P1-derived artificial chromosome (PAC)) or a viral vector (e.g., any adenoviral vectors (e.g., pSV or pCMV vectors), any retroviral vectors as described herein) and any Gateway® vectors. A vector can, e.g., include sufficient cis-acting elements for expression; other elements for expression can be supplied by the host cell or in an in vitro expression system. The term “vector” includes any genetic element (e.g., a plasmid, a transposon, a cosmid, an artificial chromosome, or a viral vector, etc.) that is capable of replicating when associated with the proper control elements. Thus, the term includes cloning and expression vectors, as well as viral vectors (e.g., an adeno-associated virus (AAV) vector, an adenovirus vector, a lentivirus vector, or a retrovirus vector).

Vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide. Skilled practitioners will be capable of selecting suitable vectors and mammalian cells for making any of the nucleic acids described herein. In some embodiments the vector is a plasmid (i.e. a circular DNA molecule that can autonomously replicate inside a cell). In some embodiments, the vector can be a cosmid (e.g., pWE and sCos series (Wahl et al. (1987), Evans et al. (1989)).

In some embodiments, the vector(s) is a viral vector (e.g., adeno-associated virus, adenovirus, lentivirus, herpes virus vector, and retrovirus). Non-limiting examples of viral vectors are described herein. In some embodiments, the vector(s) is an adeno-associated viral vector (AAV) (see, e.g., Asokan et al., Mol. Ther. 20: 699-7080, 2012). “Recombinant AAV vectors” or “rAAVs” are typically composed of, at a minimum, a transgene or a portion thereof and a regulatory sequence, and optionally 5′ and 3′ AAV inverted terminal repeats (ITRs). Such a recombinant AAV vector is packaged into a capsid and delivered to a selected target cell (e.g., an outer hair cell).

The AAV sequences of the vector typically include the cis-acting 5′ and 3′ ITR sequences (See, e.g., B. J. Carter, in “Handbook of Parvoviruses”, ed., P. Tijsser, CRC Press, pp. 155 168, 1990). Typical AAV ITR sequences are about 145 nucleotides in length. In some embodiments, at least 75% of a typical ITR sequence (e.g., at least 80%, at least 85%, at least 90%, or at least 95%) is incorporated into the AAV vector. The ability to modify these ITR sequences is within the skill of the art. (See, e.g., texts such as Sambrook et al., “Molecular Cloning. A Laboratory Manual”, 2d ed., Cold Spring Harbor Laboratory, New York, 1989; and K. Fisher et al., J Virol. 70:520 532, 1996). In some embodiments, any of the coding sequences described herein are flanked by 5′ and 3′ AAV ITR sequences in the AAV vectors. The AAV ITR sequences may be obtained from any known AAV, including presently identified AAV types.

AAV vectors as described herein may include any of the regulatory elements described herein (e.g., one or more of a promoter, one or more enhancers, one or more repressor elements, a polyA sequence, self-cleaving 2A polypeptide, and an IRES).

In some embodiments, the vector(s) is a plasmid and can include a total length of up to about 1 kb, up to about 2 kb, up to about 3 kb, up to about 4 kb, up to about 5 kb, up to about 6 kb, up to about 7 kb, up to about 8kb, up to about 9 kb, up to about 10 kb, up to about 11 kb, up to about 12 kb, up to about 13 kb, up to about 14 kb, or up to about 15 kb. In some embodiments, the vector(s) is a plasmid and can have a total length in a range of about 1 kb to about 2 kb, about 1 kb to about 3 kb, about 1 kb to about 4 kb, about 1 kb to about 5 kb, about 1 kb to about 6 kb, about 1 kb to about 7 kb, about 1 kb to about 8 kb, about 1 kb to about 9 kb, about 1 kb to about 10 kb, about 1 kb to about 11 kb, about 1 kb to about 12 kb, about 1 kb to about 13 kb, about 1 kb to about 14 kb, or about 1 kb to about 15 kb.

In some embodiments, the vector(s) is a viral vector and can have a total number of nucleotides of up to 10 kb. In some embodiments, the viral vector(s) can have a total number of nucleotides in the range of about 1 kb to about 2 kb, 1 kb to about 3 kb, about 1 kb to about 4 kb, about 1 kb to about 5 kb, about 1 kb to about 6 kb, about 1 kb to about 7 kb, about 1 kb to about 8 kb, about 1 kb to about 9 kb, about 1 kb to about 10 kb, about 2 kb to about 3 kb, about 2 kb to about 4 kb, about 2 kb to about 5 kb, about 2 kb to about 6 kb, about 2 kb to about 7 kb, about 2 kb to about 8 kb, about 2 kb to about 9 kb, about 2 kb to about 10 kb, about 3 kb to about 4 kb, about 3 kb to about 5 kb, about 3 kb to about 6 kb, about 3 kb to about 7 kb, about 3 kb to about 8 kb, about 3 kb to about 9 kb, about 3 kb to about 10 kb, about 4 kb to about 5 kb, about 4 kb to about 6 kb, about 4 kb to about 7 kb, about 4 kb to about 8 kb, about 4 kb to about 9 kb, about 4 kb to about 10 kb, about 5 kb to about 6 kb, about 5 kb to about 7 kb, about 5 kb to about 8 kb, about 5 kb to about 9 kb, about 5 kb to about 10 kb, about 6 kb to about 7 kb, about 6 kb to about 8 kb, about 6 kb to about 9 kb, about 6 kb to about 10 kb, about 7 kb to about 8 kb, about 7 kb to about 9 kb, about 7 kb to about 10 kb, about 8 kb to about 9 kb, about 8 kb to about 10 kb, or about 9 kb to about 10 kb.

In some embodiments, the vector(s) is an adeno-associated virus (AAV vector) and can include a total number of nucleotides of up to 5 kb. In some embodiments, the AAV vector(s) can include a total number of nucleotides in the range of about 1 kb to about 2 kb, about 1 kb to about 3 kb, about 1 kb to about 4 kb, about 1 kb to about 5 kb, about 2 kb to about 3 kb, about 2 kb to about 4 kb, about 2 kb to about 5kb, about 3 kb to about 4 kb, about 3 kb to about 5 kb, or about 4 kb to about 5 kb.

A variety of different methods known in the art can be used to introduce any of vectors disclosed herein into a mammalian cell (e.g., a muscle cell). Non-limiting examples of methods for introducing nucleic acid into a mammalian cell include:

lipofection, transfection (e.g., calcium phosphate transfection, transfection using highly branched organic compounds, transfection using cationic polymers, dendrimer-based transfection, optical transfection, particle-based transfection (e.g., nanoparticle transfection), or transfection using liposomes (e.g., cationic liposomes)), microinjection, electroporation, cell squeezing, sonoporation, protoplast fusion, impalefection, hydrodynamic delivery, gene gun, magnetofection, viral transfection, and nucleofection.

Skilled practitioners will appreciate that any of the vectors described herein can be introduced into a mammalian cell by, for example, lipofection, and can be stably integrated into an endogenous gene locus.

Any of the vectors described herein can further include a control sequence, e.g., a control sequence selected from the group of a transcription initiation sequence, a transcription termination sequence, a promoter sequence, an enhancer sequence, an RNA splicing sequence, a polyadenylation (polyA) sequence, and a Kozak consensus sequence. Non-limiting examples of these control sequences are described herein. In some embodiments, a promoter can be a native promoter, a constitutive promoter, an inducible promoter, and/or a tissue-specific promoter.

Some embodiments of any of the compositions and kits described herein can include any combination of the AAV vectors described herein. Some embodiments of any of the methods described herein can include the use of any combination of the AAV vectors described herein.

### Promoters

The term “promoter” means a DNA sequence recognized by enzymes/proteins in a mammalian cell required to initiate the transcription of a specific gene (e.g., a SARS CoV-2 polypeptide of fragment thereof). A promoter typically refers to, e.g., a nucleotide sequence to which an RNA polymerase and/or any associated factor binds and at which transcription is initiated. Non-limiting examples of promoters are described herein. Additional examples of promoters are known in the art.

In some embodiments, the promoter is a constitutive promoter, a muscle-specific promoter, an inducible promoter a viral promoter, a chimeric promoter, an engineered promoter, or any other type of promoter known in the art. In some embodiments, the promoter is a RNA polymerase II promoter. In some embodiments, the promoter is a RNA polymerase III promoter, including, but not limited to, a H1 promoter, a human U6 promoter, a mouse U6 promoter, or a swine U6 promoter. The promoter will generally be one that is able to promote transcription in muscle cells.

A variety of promoters are known in the art that can be used herein. Non-limiting examples of promoters that can be used herein include: human EF1a, human cytomegalovirus (CMV) (U.S. Pat. No. 5,168,062), human ubiquitin C (UBC), mouse phosphoglycerate kinase 1, polyoma adenovirus, simian virus 40 (SV40), β-globin, β-actin, α-fetoprotein, γ-globin, β-interferon, γ-glutamyl transferase, mouse mammary tumor virus (MMTV), Rous sarcoma virus, rat insulin, glyceraldehyde-3-phosphate dehydrogenase, metallothionein II (MT II), amylase, cathepsin, MI muscarinic receptor, retroviral LTR (e.g. human T-cell leukemia virus HTLV), AAV ITR, interleukin-2, collagenase, platelet-derived growth factor, adenovirus 5 E2, stromelysin, murine MX gene, glucose regulated proteins (GRP78 and GRP94), α-2-macroglobulin, vimentin, MHC class I gene H-2κ b, HSP70, proliferin, tumor necrosis factor, thyroid stimulating hormone a gene, immunoglobulin light chain, T-cell receptor, HLA DQα and DQβ, interleukin-2 receptor, MHC class II, MHC class II HLA-DRα, muscle creatine kinase, prealbumin (transthyretin), elastase I, albumin gene, c-fos, c-HA-ras, neural cell adhesion molecule (NCAM), H2B (TH2B) histone, rat growth hormone, human serum amyloid (SAA), troponin I (TN I), duchenne muscular dystrophy, human immunodeficiency virus, and Gibbon Ape Leukemia Virus (GALV) promoters. Additional examples of promoters are known in the art. See, e.g., Lodish, Molecular Cell Biology, Freeman and Company, New York 2007. In some embodiments, the promoter is the CMV immediate early promoter. In some embodiments, the promoter is a CAG promoter or a CAG/CBA promoter.

The term “constitutive” promoter refers to a nucleotide sequence that, when operably linked with a nucleic acid encoding a protein (e.g., a SARS-CoV2 polypeptide or fragment thereof), causes RNA to be transcribed from the nucleic acid in a mammalian cell under most or all physiological conditions.

Examples of constitutive promoters include, without limitation, the retroviral Rous sarcoma virus (RSV) LTR promoter, the cytomegalovirus (CMV) promoter (see, e.g., Boshart et al, Cell 41:521-530, 1985), the SV40 promoter, the dihydrofolate reductase promoter, the beta-actin promoter, the phosphoglycerol kinase (PGK) promoter, and the EF1-alpha promoter (Invitrogen).

The term “tissue-specific” or “muscle-specific” promoter refers to a promoter that is active only in certain specific cell types and/or tissues (e.g., transcription of a specific gene occurs only within cells expressing transcription regulatory proteins that bind to the tissue-specific promoter). In some embodiments, the regulatory sequences impart tissue-specific gene expression capabilities. In some cases, the tissue-specific regulatory sequences bind tissue-specific transcription factors that induce transcription in a tissue-specific manner. In some embodiments, the promoter is optimized for expression in the muscle. In some embodiments, the promoter is a muscle-specific promoter. For example, muscle-specific promoters include, without limitation, skeletal β-actin, myosin light chain 2A, dystrophin, muscle creatine kinase, SPc-512, and synthetic muscle promoters muscle promoters with activities higher than naturally-occurring promoters (see Li et al., Nat. Biotech., 17:241-245 (1999)).

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech, and Ariad. Additional examples of inducible promoters are known in the art.

Examples of inducible promoters regulated by exogenously supplied compounds include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al, Proc. Natl. Acad. Sci. U.S.A. 93:3346-3351, 1996), the tetracycline-repressible system (Gossen et al, Proc. Natl. Acad. Sci. U.S.A. 89:5547-5551, 1992), the tetracycline-inducible system (Gossen et al, Science 268:1766-1769, 1995, see also Harvey et al, Curr. Opin. Chem. Biol. 2:512-518, 1998), the RU486-inducible system (Wang et al, Nat. Biotech. 15:239-243, 1997) and Wang et al, Gene Ther. 4:432-441, 1997), and the rapamycin-inducible system (Magari et al. J. Clin. Invest. 100:2865-2872, 1997).

### Enhancers and 5′ Cap

In some instances, a vector can include a promoter sequence and/or an enhancer sequence. The term “enhancer” refers to a nucleotide sequence that can increase the level of transcription of a nucleic acid encoding a protein of interest. Enhancer sequences (50-1500 basepairs in length) generally increase the level of transcription by providing additional binding sites for transcription-associated proteins (e.g., transcription factors). In some embodiments, an enhancer sequence is found within an intronic sequence. Unlike promoter sequences, enhancer sequences can act at much larger distance away from the transcription start site (e.g., as compared to a promoter). Non-limiting examples of enhancers include a RSV enhancer, a CMV enhancer, and a SV40 enhancer.

### Poly(A) Sequences

In some embodiments, any of the vectors provided herein can include a poly(A) sequence. Most nascent eukaryotic mRNAs possess a poly(A) tail at their 3′ end which is added during a complex process that includes cleavage of the primary transcript and a coupled polyadenylation reaction (see, e.g., Proudfoot et al., Cell 108:501-512, 2002). The poly(A) tail confers mRNA stability and transferability (Molecular Biology of the Cell, Third Edition by B. Alberts et al., Garland Publishing, 1994). In some embodiments, the poly(A) sequence is positioned 3′ to the nucleic acid sequence encoding the C-terminus of the SARS-CoV2 polypeptide.

As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. The 3′ poly(A) tail is a long sequence of adenine nucleotides (e.g., 50, 60, 70, 100, 200, 500, 1000, 2000, 3000, 4000, or 5000) added to the pre-mRNA through the action of an enzyme, polyadenylate polymerase. In higher eukaryotes, the poly(A) tail is added onto transcripts that contain a specific sequence, the polyadenylation signal or “poly(A) sequence.” The poly(A) tail and the protein bound to it aid in protecting mRNA from degradation by exonucleases. Polyadenylation is also important for transcription termination, export of the mRNA from the nucleus, and translation. Polyadenylation occurs in the nucleus immediately after transcription of DNA into RNA, but additionally can also occur later in the cytoplasm. After transcription has been terminated, the mRNA chain is cleaved through the action of an endonuclease complex associated with RNA polymerase. The cleavage site is usually characterized by the presence of the base sequence AAUAAA near the cleavage site. After the mRNA has been cleaved, adenosine residues are added to the free 3′ end at the cleavage site.

As used herein, a “poly(A) sequence” is a sequence that triggers the endonuclease cleavage of an mRNA and the additional of a series of adenosines to the 3′ end of the cleaved mRNA.

There are several poly(A) sequences that can be used, including those derived from bovine growth hormone (bgh) (Woychik et al., Proc. Natl. Acad. Sci. U.S.A. 81(13):3944-3948, 1984; U.S. Pat. No. 5,122,458), mouse-β-globin, mouse-α-globin (Orkin et al., EMBO J. 4(2):453-456, 1985; Thein et al., Blood 71(2):313-319, 1988), human collagen, polyoma virus (Batt et al., Mol. Cell Biol. 15(9):4783-4790, 1995), the Herpes simplex virus thymidine kinase gene (HSV TK), IgG heavy-chain gene polyadenylation signal (US 2006/0040354), human growth hormone (hGH) (Szymanski et al., Mol. Therapy 15(7):1340-1347, 2007), the group of SV40 poly(A) sites, such as the SV40 late and early poly(A) site (Schek et al., Mol. Cell Biol. 12(12):5386-5393, 1992).

The poly(A) sequence can a sequence of AATAAA. The AATAAA sequence may be substituted with other hexanucleotide sequences with homology to AATAAA which are capable of signaling polyadenylation, including ATTAAA, AGTAAA, CATAAA, TATAAA, GATAAA, ACTAAA, AATATA, AAGAAA, AATAAT, AAAAAA, AATGAA, AATCAA, AACAAA, AATCAA, AATAAC, AATAGA, AATTAA, or AATAAG (see, e.g., WO 06/12414).

In some embodiments, the poly(A) sequence can be a synthetic polyadenylation site (see, e.g., the pCl-neo expression vector of Promega which is based on Levitt et al, Genes Dev. 3(7):1019-1025, 1989). In some embodiments, the poly(A) sequence is the polyadenylation signal of soluble neuropilin-1 (sNRP) (AAATAAAATACGAAATG) (see, e.g., WO 05/073384). Additional examples of poly(A) sequences are known in the art.

### Internal Ribosome Entry Site (IRES)

In some embodiments, a vector encoding a SARS-CoV-2 polypeptide or fragment thereof can include a polynucleotide internal ribosome entry site (IRES). An IRES sequence is used to produce more than one polypeptide from a single gene transcript. An IRES forms a complex secondary structure that allows translation initiation to occur from any position with an mRNA immediately downstream from where the IRES is located (see, e.g., Pelletier and Sonenberg, Mol. Cell. Biol. 8(3):1103-1112, 1988).

There are several IRES sequences known to those in skilled in the art, including those from, e.g., foot and mouth disease virus (FMDV), encephalomyocarditis virus (EMCV), human rhinovirus (HRV), cricket paralysis virus, human immunodeficiency virus (HIV), hepatitis A virus (HAV), hepatitis C virus (HCV), and poliovirus (PV). See e.g., Alberts, Molecular Biology of the Cell, Garland Science, 2002; and Hellen et al., Genes Dev. 15(13):1593-612, 2001.

In some embodiments, the IRES sequence that is incorporated into the vector that encodes the SARS-CoV-2 polypeptide or fragment thereof is the foot and mouth disease virus (FMDV). The Foot and Mouth Disease Virus 2A sequence is a small peptide (approximately 18 amino acids in length) that has been shown to mediate the cleavage of polyproteins (Ryan, M D et al., EMBO 4:928-933, 1994; Mattion et al., J. Virology 70:8124-8127, 1996; Furler et al., Gene Therapy 8:864-873, 2001; and Halpin et al., Plant Journal 4:453-459, 1999). The cleavage activity of the 2A sequence has previously been demonstrated in artificial systems including plasmids and gene therapy vectors (AAV and retroviruses) (Ryan et al., EMBO 4:928-933, 1994; Mattion et al., J. Virology 70:8124-8127, 1996; Furler et al., Gene Therapy 8:864-873, 2001; and Halpin et al., Plant Journal 4:453-459, 1999; de Felipe et al., Gene Therapy 6:198-208, 1999; de Felipe et al., Human Gene Therapy 11:1921-1931, 2000; and Klump et al., Gene Therapy 8:811-817, 2001).

### Reporter Sequences

Any of the vectors provided herein can optionally include a sequence encoding a reporter protein (“a reporter sequence”). Non-limiting examples of reporter sequences include DNA sequences encoding: a beta-lactamase, a beta-galactosidase (LacZ), an alkaline phosphatase, a thymidine kinase, a green fluorescent protein (GFP), a red fluorescent protein, an mCherry fluorescent protein, a yellow fluorescent protein, a chloramphenicol acetyltransferase (CAT), and a luciferase. Additional examples of reporter sequences are known in the art. When associated with regulatory elements which drive their expression, the reporter sequence can provide signals detectable by conventional means, including enzymatic, radiographic, colorimetric, fluorescence, or other spectrographic assays; fluorescent activating cell sorting (FACS) assays; immunological assays (e.g., enzyme linked immunosorbent assay (ELISA), radioimmunoassay (RIA), and immunohistochemistry).

In some embodiments, the reporter sequence is the LacZ gene, and the presence of a vector carrying the LacZ gene in a mammalian cell is detected by assays for beta-galactosidase activity. In other embodiments, the reporter is a fluorescent protein (e.g., green fluorescent protein) or luciferase, the presence of a vector carrying the fluorescent protein or luciferase in a mammalian cell may be measured by fluorescent techniques (e.g., fluorescent microscopy or FACS) or light production in a luminometer (e.g., a spectrophotometer or an IVIS imaging instrument). In some embodiments, the reporter sequence can be used to verify the tissue-specific targeting capabilities and tissue-specific promoter regulatory activity of any of the vectors described herein.

### Flanking Regions Untranslated Regions (UTRs)

In some embodiments, any of the vectors described herein can include an untranslated region. In some embodiments, a vector can includes a 5′ UTR or a 3′ UTR. Untranslated regions (UTRs) of a gene are transcribed but not translated. The 5′ UTR starts at the transcription start site and continues to the start codon but does not include the start codon. The 3′ UTR starts immediately following the stop codon and continues until the transcriptional termination signal. There is growing body of evidence about the regulatory roles played by the UTRs in terms of stability of the nucleic acid molecule and translation. The regulatory features of a UTR can be incorporated into any of the vectors, compositions, kits, or methods as described herein to enhance the stability of a SARS-CoV-2 polypeptide.

Natural 5′ UTRs include a sequence that plays a role in translation initiation. They harbor signatures like Kozak sequences, which are commonly known to be involved in the process by which the ribosome initiates translation of many genes. Kozak sequences have the consensus sequence CCR(A/G)CCAUGG, where R is a purine (A or G) three bases upstream of the start codon (AUG), which is followed by another “G”. The 5′ UTR have also been known, e.g., to form secondary structures that are involved in elongation factor binding.

For example, in some embodiments, a 5′ UTR is included in any of the vectors described herein. Non-limiting examples of 5′ UTRs including those from the following genes: albumin, serum amyloid A, Apolipoprotein A/B/E, transferrin, alpha fetoprotein, erythropoietin, and Factor VIII, can be used to enhance expression of a nucleic acid molecule, such as a mRNA.

In some embodiments, a 5′ UTR from a mRNA that is transcribed by a cell in the muscle cell can be included in any of the vectors, compositions, kits, and methods described herein.

3′ UTRs are known to have stretches of adenosines and uridines embedded in them. These AU-rich signatures are particularly prevalent in genes with high rates of turnover. Based on their sequence features and functional properties, the AU-rich elements (AREs) can be separated into three classes (Chen et al., Mol. Cell. Biol. 15:5777-5788, 1995; Chen et al., Mol. Cell Biol. 15:2010-2018, 1995): Class I AREs contain several dispersed copies of an AUUUA motif within U-rich regions. For example, c-Myc and MyoD mRNAs contain class I AREs. Class II AREs possess two or more overlapping UUAUUUA(U/A) (U/A) nonamers. GM-CSF and TNF-alpha mRNAs are examples that contain class II AREs. Class III AREs are less well defined. These U-rich regions do not contain an AUUUA motif. Two well-studied examples of this class are c-Jun and myogenin mRNAs.

Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

In some embodiments, the introduction, removal, or modification of 3′ UTR AREs can be used to modulate the stability of an mRNA encoding a SARS-CoV-2 polypeptide. In other embodiments, AREs can be removed or mutated to increase the intracellular stability and thus increase translation and production of any of the polypeptides described herein.

In other embodiments, non-UTR sequences may be incorporated into the 5′ or 3′ UTRs. In some embodiments, introns or portions of intron sequences may be incorporated into the flanking regions of the polynucleotides in any of the vectors, compositions, kits, and methods provided herein. Incorporation of intronic sequences may increase protein production as well as mRNA levels.

### Methods

Also provided herein are methods of inducing at least partial immunity to a coronavirus in a subject that includes administering to the subject a therapeutically effective amount of any of the AAVs described herein, any of the vectors described herein, any of the pharmaceutical compositions described herein and any of the vaccine compositions described herein. In some embodiments, the method of inducing at least partial immunity to a coronavirus in a subject includes administering an adjuvant (e.g., a CpG adjuvant).

Also provided herein are methods of inducing at least partial humoral immunity to a coronavirus in a subject that includes administering to the subject a therapeutically effective amount of any of the AAVs described herein, any of the vectors described herein, any of the pharmaceutical compositions described herein and any of the vaccine compositions described herein. In some embodiments, the method of inducing at least partial humoral immunity to a coronavirus in a subject further includes administering an adjuvant (e.g., a CpG adjuvant).

Also provided herein are methods of inducing at least partial cellular immunity to a coronavirus in a subject that includes administering to the subject a therapeutically effective amount of any of the AAVs described herein, any of the vectors described herein, any of the pharmaceutical compositions described herein and any of the vaccine compositions described herein. In some embodiments, the method of inducing at least partial cellular immunity to a coronavirus in a subject further includes administering an adjuvant (e.g., a CpG adjuvant).

Also provided herein are methods of increasing the titer of antibodies in a subject that specifically bind to a SARS-CoV2 polypeptide or a fragment thereof that include administering to the subject a therapeutically effective amount of any of the AAVs described herein, any of the vectors described herein, any of the pharmaceutical compositions described herein and any of the vaccine compositions described herein. In some embodiments, the method of increasing the titer of antibodies in a subject that specifically bind to a SARS-CoV2 polypeptide further includes administering an adjuvant (e.g., a CpG adjuvant).

Also provided herein are methods of decreasing the rate of infection of a coronavirus in a subject that include administering to the subject a therapeutically effective amount of any of the AAVs described herein, any of the vectors described herein, any of the pharmaceutical compositions described herein and any of the vaccine compositions described herein. In some embodiments, the method of decreasing the rate of infection of a coronavirus in a subject further includes administering an adjuvant (e.g., a CpG adjuvant).

In some embodiments of any of these methods described herein, the subject has been identified as not having previously had a coronavirus infection. In some embodiments of any of these methods described herein, prior to administration of any of the AAVs, vectors, pharmaceutical compositions or vaccine compositions, the subject has been identified as not having a significant titer of antibodies that bind specifically to the SARS-CoV-2 polypeptide or a fragment thereof.

In some embodiments of any of the methods described herein, the coronavirus is a 229E (alpha coronavirus), NL63 (alpha coronavirus), OC43 (beta coronavirus), HKU1 (beta coronavirus), MERS-CoV (beta coronavirus that causes Middle East Respiratory Syndrome (MERS)), SARS-CoV (beta coronavirus that causes severe acute respiratory syndrome (SARS)), or SARS-CoV-2 (novel coronavirus that causes coronavirus disease 2019 (COVID-19)). In some embodiments, the coronavirus is SARS-CoV-2.

In some embodiments of any of the methods described herein, the subject has previously been identified as having one or more medical conditions selected from the group consisting of: chronic lung disease, moderate asthma, severe asthma, heart conditions, diabetes, obesity, liver disease, chronic kidney disease, and a weakened or suppressed immune system. In some embodiments, wherein the subject having a weakened or suppressed immune system is a subject receiving a cancer treatment, a smoker, a subject who is a transplant recipient, a subject having HIV or AIDS, or a subject receiving a corticosteroid or any other immunosuppressant drug. In some embodiments, the subject having a weakened or suppressed immune system is an elderly subject.

Methods of detecting the presence of an antibody (e.g., an antibody that specifically binds to a SARS-CoV-2 polypeptide) in a subject are known in the art. In some embodiments, the presence of antibody can be detected directly (e.g., detecting mRNA). Non-limiting examples of techniques that can be used to the presence of an antibody (e.g., an antibody that specifically binds to a SARS-CoV-2 polypeptide) include: ELISA, qRT-PCR, next generation sequence, antibody profiling, real-time PCR, Western blotting, immunoprecipitation, immunohistochemistry, or immunofluorescence.

In some embodiments, the method of detecting the presence of a coronavirus (e.g., SARS-CoV-2) in a subject includes obtaining a sample (e.g., any of the exemplary samples described herein or known in the art) from a subject. Non-limiting examples of the types of samples taken from a subject can include a nasopharyngeal (NP) specimen, oropharyngeal (OP) specimen, nasal mid-turbinate swab, anterior nares (nasal swab) specimen, nasopharyngeal wash/aspirate or nasal wash/aspirate (NW) specimen, blood specimen, saliva specimen, and a fecal specimen.

Methods of detecting the presence of a coronavirus (e.g., SARS-CoV-2) in a subject are known in the art. In some embodiments, the presence of a coronavirus can be detected by viral RNA (e.g., detecting viral RNA using any of the exemplary techniques described herein or know in the art). Non-limiting examples of techniques that can be used to detect the presence of coronavirus (e.g., a SARS-CoV-2 coronavirus) include: ELISA, qRT-PCR, next generation sequencing, antibody profiling, real-time PCR, Western blotting, immunoprecipitation, immunohistochemistry, nucleic acid detection using Cas13 (see e.g., Ackerman et al., Nat., 582: 277-282 (2020)), nucleic acid detection using CRISPR-Cas12 (see, e.g., Broughton et al., Nat. Biotech., 38: 870-874 (2020)), nucleic acid detection using loop-mediated isothermal amplification (LAMP), RT-LAMP and LAMP-sequencing (see e.g., Thi et al., Sci, Transl. Med., 12: (2020)), any nucleic acid kit that uses recombinase polymerase amplification (RPA) or immunofluorescence. In some embodiments, the method of detecting the presence of a coronavirus includes detecting viral shedding (e.g., as measured by RT-qPCR). In some embodiments, the presence of a coronavirus can be detected by antibodies that specifically bind to a SARS-CoV-2 glycoprotein polypeptide. Methods of detecting antibodies are well-known in the art and include but are not limited to enzyme linked immunosorbent assay (ELISA), fluorescent activated cell sorting (FACS), Western blotting, immunoprecipitation, immunofluorescence, mass spectrometry (LC-MS, Inductively Coupled Plasma Mass Spectrometry (ICP-MS), MS based methods (LC-MS/MS), and electrochemicalluminecence immunoassay.

### Pharmaceutical Compositions and Kits

In some embodiments, any of the pharmaceutical compositions or vaccine compositions described herein can further include one or more agents that promote the entry of a nucleic acid or any of the vectors described herein into a mammalian cell (e.g., a liposome or cationic lipid).

In some embodiments, any of the AAVs or vectors described herein can be formulated using natural and/or synthetic polymers. Non-limiting examples of polymers that may be included in any of the compositions described herein can include, but are not limited to, DYNAMIC POLYCONJUGATE® (Arrowhead Research Corp., Pasadena, Calif.), formulations from Minis Bio (Madison, Wis.) and Roche Madison (Madison, Wis.), PhaseRX polymer formulations such as, without limitation, SMARTT POLYMER TECHNOLOGY® (PhaseRX, Seattle, Wash.), DMRI/DOPE, poloxamer, VAXFECTIN® adjuvant from Vical (San Diego, Calif.), chitosan, cyclodextrin from Calando Pharmaceuticals (Pasadena, Calif.), dendrimers and poly (lactic-co-glycolic acid) (PLGA) polymers, RONDEL™ (RNAi/Oligonucleotide Nanoparticle Delivery) polymers (Arrowhead Research Corporation, Pasadena, Calif.), and pH responsive co-block polymers, such as, but not limited to, those produced by PhaseRX (Seattle, Wash.). Many of these polymers have demonstrated efficacy in delivering oligonucleotides in vivo into a mammalian cell (see, e.g., deFougerolles, Human Gene Ther. 19:125-132, 2008; Rozema et al., Proc. Natl. Acad. Sci. U.S.A. 104:12982-12887, 2007; Rozema et al., Proc. Natl. Acad. Sci. U.S.A. 104:12982-12887, 2007; Hu-Lieskovan et al., Cancer Res. 65:8984-8982, 2005; Heidel et al., Proc. Natl. Acad. Sci. U.S.A. 104:5715-5721, 2007).

In some embodiments, the compositions described herein can be, e.g., a pharmaceutical composition. A pharmaceutical composition can include any of the compositions described herein and one or more pharmaceutically or physiologically acceptable carriers, diluents, or excipients. Such compositions may include one or more buffers, such as neutral-buffered saline, phosphate-buffered saline, and the like; one or more carbohydrates, such as glucose, mannose, sucrose, and dextran; mannitol; one or more proteins, polypeptides, or amino acids, such as glycine; one or more antioxidants; one or more chelating agents, such as EDTA or glutathione; and/or one or more preservatives.

In some embodiments, the compositions described herein can be, e.g., a vaccine composition. A vaccine composition can include any of the compositions described herein and one or more adjuvants, carriers, diluents, or excipients. In some embodiments, the vaccine compositions include formulations that contain one or more buffers, such as neutral-buffered saline, phosphate-buffered saline, and the like; one or more carbohydrates, such as glucose, mannose, sucrose, and dextran; mannitol; one or more proteins, polypeptides, or amino acids, such as glycine; one or more antioxidants; one or more chelating agents, such as EDTA or glutathione; and/or one or more preservatives.

In some embodiments, the composition (e.g., pharmaceutical and/or vaccine compositions) includes a pharmaceutically acceptable carrier (e.g., phosphate buffered saline, saline, or bacteriostatic water). Upon formulation, solutions will be administered in a manner compatible with the dosage formulation and in such amount as is therapeutically effective. The formulations are easily administered in a variety of dosage forms such as injectable solutions, injectable gels, drug-release capsules, and the like.

As used herein, the term “pharmaceutically acceptable carrier” includes solvents, dispersion media, coatings, antibacterial agents, antifungal agents, and the like that are compatible with pharmaceutical administration. Supplementary active compounds can also be incorporated into any of the compositions described herein.

In some embodiments, a single dose of any of the compositions (e.g., pharmaceutical and/or vaccine compositions) described herein can include a total sum amount AAVs or vectors of at least 1 ng, at least 2 ng, at least 4 ng, about 6 ng, about 8 ng, at least 10 ng, at least 20 ng, at least 30 ng, at least 40 ng, at least 50 ng, at least 60 ng, at least 70 ng, at least 80 ng, at least 90 ng, at least 100 ng, at least 200 ng, at least 300 ng, at least 400 ng, at least 500 ng, at least 1 μg, at least 2 μg, at least 4 μg, at least 6 μg, at least 8 μg, at least 10 μg, at least 12 μg, at least 14 μg, at least 16 μg, at least 18 μg, at least 20 μg, at least 22 μg, at least 24 μg, at least 26 μg, at least 28 μg, at least 30 μg at least 32 μg, at least 34 μg, at least 36 μg, at least 38 μg, at least 40 μg, at least 42 μg, at least 44 μg, at least 46 μg, at least 48 μg, at least 50 μg, at least 52 μg, at least 54 μg, at least 56 μg, at least 58 μg, at least 60 μg, at least 62 μg, at least 64 μg, at least 66 μg, at least 68 μg, at least 70 μg, at least 72 μg, at least 74 μg, at least 76 μg, at least 78 μg, at least 80 μg, at least 82 μg, at least 84 μg, at least 86 μg, at least 88 μg, at least 90 μg, at least 92 μg, at least 94 μg, at least 96 μg, at least 98 μg, at least 100 μg, at least 102 μg, at least 104 μg, at least 106 μg, at least 108 μg, at least 110 μg, at least 112 μg, at least 114 μg, at least 116 μg, at least 118 μg, at least 120 μg, at least 122 μg, at least 124 μg, at least 126 μg, at least 128 μg, at least 130 μg at least 132 μg, at least 134 μg, at least 136 μg, at least 138 μg, at least 140 μg, at least 142 μg, at least 144 μg, at least 146 μg, at least 148 μg, at least 150 μg, at least 152 μg, at least 154 μg, at least 156 μg, at least 158 μg, at least 160 μg, at least 162 μg, at least 164 μg, at least 166 μg, at least 168 μg, at least 170 μg, at least 172 μg, at least 174 μg, at least 176 μg, at least 178 μg, at least 180 μg, at least 182 μg, at least 184 μg, at least 186 μg, at least 188 μg, at least 190 μg, at least 192 μg, at least 194 μg, at least 196 μg, at least 198 μg, or at least 200 μg, e.g., in a buffered solution.

The compositions (e.g., pharmaceutical and/or vaccine compositions) provided herein can be, e.g., formulated to be compatible with their intended route of administration. A non-limiting example of an intended route of administration is local administration (intravenous or injection (e.g., intramuscular injection).

In some embodiments, the therapeutic compositions are formulated to include a lipid nanoparticle. In some embodiments, the therapeutic compositions are formulated to include a polymeric nanoparticle. In some embodiments, the therapeutic compositions are formulated to include a mini-circle DNA. In some embodiments, the therapeutic compositions are formulated to include a CELiD DNA. In some embodiments, the therapeutic compositions are formulated to include a synthetic perilymph solution. An exemplary synthetic perilymph solution includes 20-200 mM NaCl; 1-5 mM KCl; 0.1-10 mM CaCl₂; 1-10 mM glucose; 2-50 mM HEPES, having a pH of between about 6 and about 9.

Also provided are kits including any of the AAVs, vectors, or compositions (e.g., pharmaceutical and/or vaccine compositions) described herein. In some embodiments, a kit can include a solid composition (e.g., a lyophilized composition including the AAVs, vectors, or pharmaceutical and/or vaccine compositions described herein) and a liquid for solubilizing the lyophilized composition. In some embodiments, a kit can include a pre-loaded syringe including any of the compositions AAVs, vectors, or pharmaceutical and/or vaccine compositions described herein.

In some embodiments, the kit includes a vial including any of the compositions described herein (e.g., formulated as an aqueous composition, e.g., an aqueous pharmaceutical composition or vaccine composition).

In some embodiments, the kits can include instructions for performing any of the methods described herein.

### EXAMPLES

### Example 1

### Construction of an AAV Vector Containing a SARS-CoV-2 S2 Domain Polypeptide or SARS-CoV-2 Nucleocapsid Polypeptide

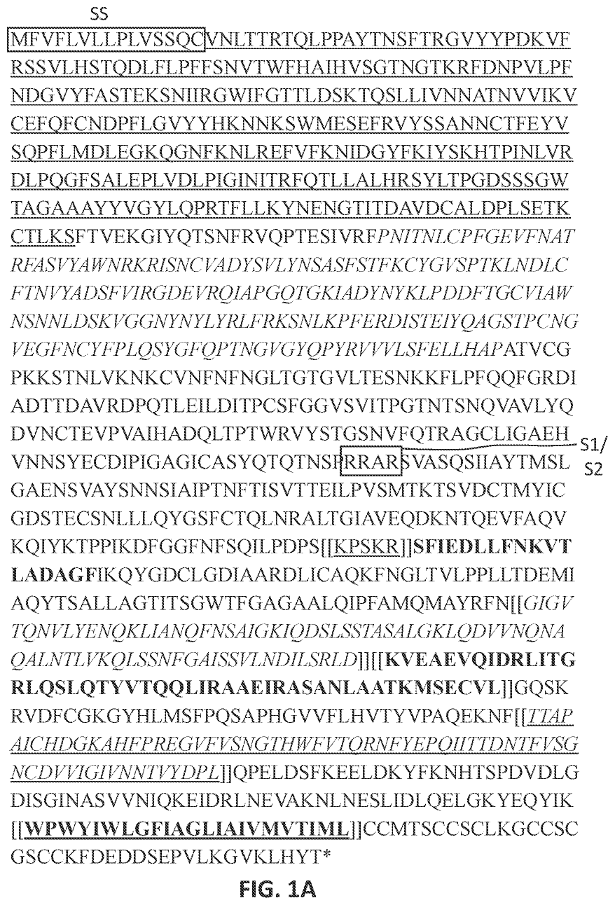

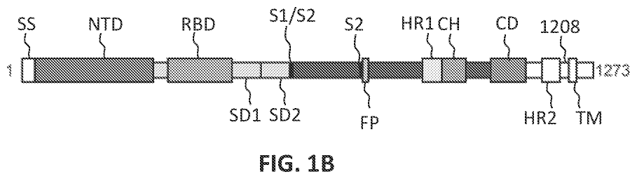

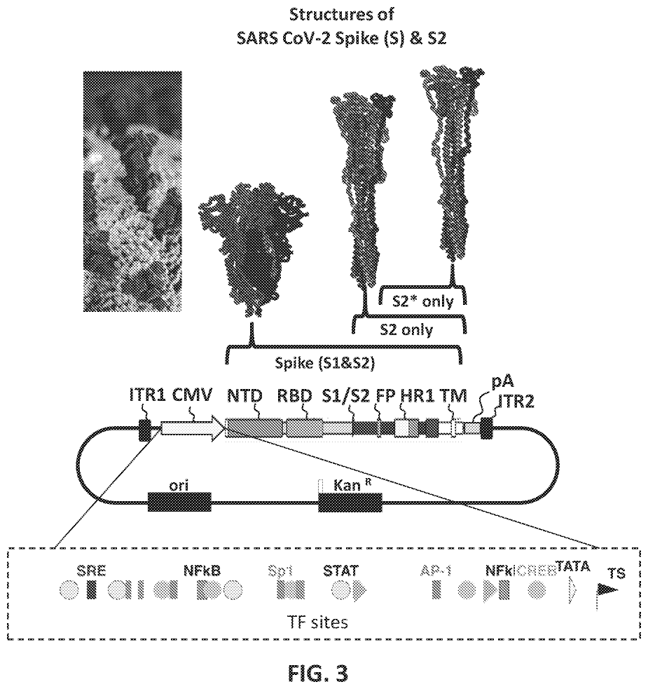

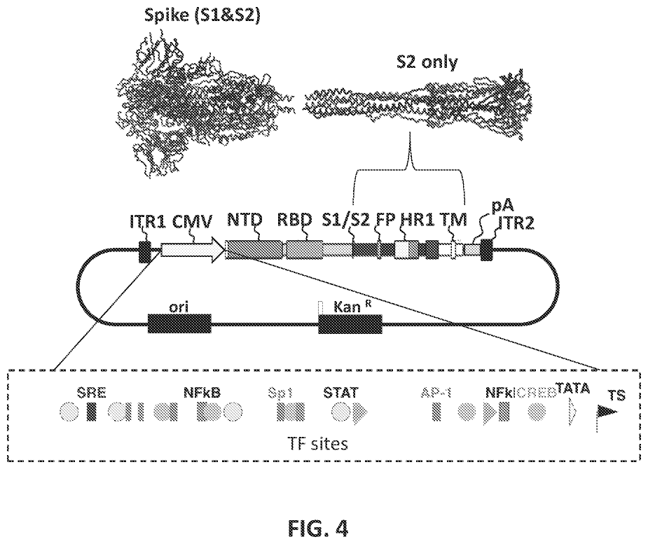

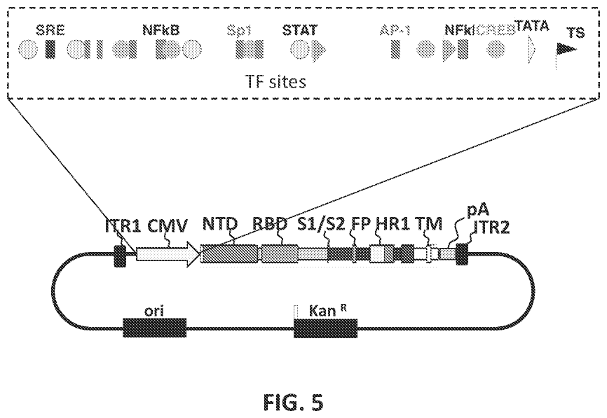

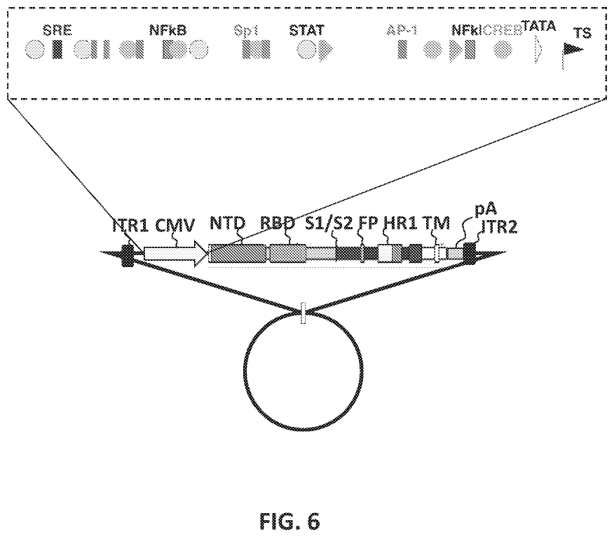

Construction of an AAV vector was performed by subcloning a nucleic acid sequence encoding a SARS-CoV-2 S2 domain (SEQ ID NO: 2 or 3) or a nucleic acid sequence encoding a SARS-CoV-2 nucleocapsid polypeptide (SEQ ID NO: 5) into an AAV vector. See FIGS. 1-6.

### Example 2

### Administration of a Therapeutically Effective Amount of an AAV to Treat a Coronavirus Infection or Prevent a Future Coronavirus Infection in a Human Subject

A human subject identified as having a coronavirus infection or at increased risk of developing a coronavirus infection is administered a therapeutically effective amount of a pharmaceutical composition comprising an AAV vector in order to treat a coronavirus infection or prevent a future coronavirus infection.

In at least some cases, a dosage of the pharmaceutical composition comprising an AAV vector that includes a nucleic acid sequence encoding a SARS-CoV-2 S glycoprotein polypeptide (SEQ ID NO: 1) is administered via intramuscular injection to the human subject. In at least some cases, the administration of the pharmaceutical composition can result an increase in titer of antibodies that specifically bind to the SARS-CoV-2 polypeptide or the fragment thereof in the subject. In at least some of the cases, the administration of the pharmaceutical composition can result in a decrease in the rate of infection of the coronavirus in the subject. In at least some of the cases, the administration of the pharmaceutical composition can result in at least partial immunity to the coronavirus due to humoral immunity to the coronavirus. In at least some of the cases, the administration of the pharmaceutical composition can result in robust T-cell mediated immunity to the coronavirus.

In at least some cases, prior to, or after, administration of the pharmaceutical composition, a sample (e.g., a blood sample, a fluid sample, or a tissue sample) taken from the human subject is assessed for the presence of a coronavirus infection (e.g., determination of viral titer) or antibodies that specifically bind to the SARS-CoV-2 glycoprotein polypeptide. The presence of or past exposure to coronavirus (e.g., SARS-CoV-2) is determined using quantitative RT-PCR to detect viral RNA and/or an ELISA assay to detect antibodies that bind specifically to the SARS-CoV-2 glycoprotein polypeptide. When SARS-CoV-2 and/or antibodies that specifically bind the SARS-CoV-2 glycoprotein polypeptide are present above a given threshold (e.g., for qRT-PCR, identified as being above a certain threshold cycle (Ct), or for antibodies that specifically bind the SARS-CoV-2 glycoprotein polypeptide, identified as having a significant titer of antibodies that bind specifically to a SARS-CoV-2 polypeptide), the patient is identified as having, or as having previously had, a SARS-CoV-2 infection. In at least some cases, a second dosage of the pharmaceutical composition comprising an AAV vector that includes a nucleic acid sequence encoding a SARS-CoV-2 S glycoprotein polypeptide (SEQ ID NO: 1) is administered via intramuscular injection to the human subject. In at least some cases, the second dosage is administered to the human subject based on, following administration of the first dose, the absence of a significant titer of antibodies that bind specifically to a SARS-CoV-2 glycoprotein polypeptide.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

What is claimed is:

1. An adeno-associated virus (AAV) vector comprising a sequence encoding a SARS-CoV-2 polypeptide or a fragment thereof.

2. The AAV vector of claim 1, wherein the SARS-CoV-2 polypeptide is a SARS-CoV-2 S glycoprotein polypeptide or a fragment thereof.

3. The AAV vector of claim 2, wherein the S glycoprotein polypeptide or the fragment thereof comprises a Si domain or a fragment thereof.

4. The AAV vector of claim 2, wherein the S glycoprotein polypeptide or the fragment thereof comprises, consists essentially, or consists of a S2 domain or a fragment thereof.

5. The AAV vector of claim 4, wherein the S2 domain or the fragment thereof comprises, consists essentially, or consists of SEQ ID NO: 2 or SEQ ID NO: 3.

6. The AAV vector of any one of claims 2-5, further comprising a sequence encoding a transmembrane domain, wherein the sequence encoding the transmembrane domain is operably linked to the sequence encoding the S glycoprotein polypeptide or the fragment thereof.

7. The AAV vector of claim 6, wherein the transmembrane domain comprises a sequence of SEQ ID NO: 4 or a fragment thereof.

8. The AAV vector of claim 1, wherein the SARS-CoV-2 polypeptide or the fragment thereof is a nucleocapsid protein or a fragment thereof.

9. The AAV vector of claim 8, wherein the nucleocapsid polypeptide or the fragment thereof comprises a sequence of SEQ ID NO: 5 or a fragment thereof.

10. The AAV vector of claim 1, wherein the SARS-CoV-2 polypeptide or the fragment thereof is an envelope polypeptide or a fragment thereof.

11. The AAV vector of claim 1, wherein the SARS-CoV-2 polypeptide or the fragment thereof is a membrane polypeptide or a fragment thereof.

12. The AAV vector of any one of claims 1-11, further comprising a sequence encoding a signal sequence, wherein the sequence encoding the signal sequence is operatively linked to the sequence encoding the SARS CoV-2 polypeptide or the fragment thereof.

13. The AAV vector of claim 12, wherein the signal sequence comprises a sequence of SEQ ID NO: 6.

14. The AAV vector of any one of claims 1-13, further comprises a promoter operatively linked to the sequence encoding the SARS CoV-2 polypeptide or the fragment thereof.

15. The AAV vector of claim 14, wherein the promoter is a muscle-specific promoter.

16. The AAV vector of claim 15, wherein the muscle-specific promoter is selected from the group consisting of: skeletal β-actin, myosin light chain 2A, dystrophin, muscle creatine kinase, SPc-512, and synthetic muscle promoters.

17. The AAV vector of claim 14, wherein the promoter is selected from the group consisting of: CMV, RSV, SV40, β-actin, PGK, and EF1 promoters.

18. The AAV vector of any one of claims 1-17, wherein the AAV vector comprises an AAV serotype 6 (AAV6) capsid protein.

19. The AAV vector of any one of claims 1-17, wherein the AAV vector comprises an AAV serotype 9 (AAV9) capsid protein.

20. The AAV vector of any one of claims 1-17, wherein the AAV vector comprises an Anc80, Anc80Lib, Anc 81, Anc82, Anc83, Anc84, Anc110, Anc113, Anc126, Anc127, or another phylogenetically related AAV capsid protein.

21. A pharmaceutical composition comprising an AAV vector of any one of claims 1-20.

22. The pharmaceutical composition of claim 21, further comprising an adjuvant.

23. The pharmaceutical composition of claim 22, wherein the adjuvant is a CpG adjuvant.

24. A vaccine composition comprising an AAV vector of any one of claims 1-20.

25. The vaccine composition of claim 24, further comprising an adjuvant.

26. The vaccine composition of claim 25, wherein the adjuvant is a CpG adjuvant.

27. A vector comprising a sequence encoding a SARS-CoV-2 polypeptide or a fragment thereof.

28. The vector of claim 27, wherein the SARS-CoV-2 polypeptide is a SARS-CoV-2 S glycoprotein polypeptide or a fragment thereof.

29. The vector of claim 28, wherein the S glycoprotein polypeptide or the fragment thereof comprises a Si domain or a fragment thereof.

30. The vector of claim 28, wherein the S glycoprotein polypeptide or the fragment thereof comprises, consists essentially, or consists of a S2 domain or a fragment thereof.

31. The vector of claim 30, wherein the S2 domain or the fragment thereof comprises, consists essentially, or consists of SEQ ID NO: 2 or SEQ ID NO: 3.

32. The vector of any one of claims 28-31, further comprising a sequence encoding a transmembrane domain, wherein the sequence encoding the transmembrane domain is operably linked to the sequence encoding the S glycoprotein polypeptide or the fragment thereof.

33. The vector of claim 32, wherein the transmembrane domain comprises a sequence of SEQ ID NO: 4 or a fragment thereof.

34. The vector of claim 27, wherein the SARS-CoV-2 polypeptide or the fragment thereof is a nucleocapsid protein or a fragment thereof.

35. The vector of claim 34, wherein the nucleocapsid polypeptide or the fragment thereof comprises a sequence of SEQ ID NO: 5 or a fragment thereof.

36. The vector of claim 27, wherein the SARS-CoV-2 polypeptide or the fragment thereof is an envelope polypeptide or a fragment thereof.

37. The vector of claim 27, wherein the SARS-CoV-2 polypeptide or the fragment thereof is a membrane polypeptide or a fragment thereof.

38. The vector of any one of claims 27-37, further comprising a sequence encoding a signal sequence, wherein the sequence encoding the signal sequence is operatively linked to the sequence encoding the SARS CoV-2 polypeptide or the fragment thereof.

39. The vector of claim 38, wherein the signal sequence comprises a sequence of SEQ ID NO: 6.

40. The vector of any one of claims 27-39, further comprises a promoter operatively linked to the sequence encoding the SARS CoV-2 polypeptide or the fragment thereof.

41. The vector of claim 40, wherein the promoter is a muscle-specific promoter.

42. The vector of claim 41, wherein the muscle-specific promoter is selected from the group consisting of: skeletal β-actin, myosin light chain 2A, dystrophin, muscle creatine kinase, SPc-512, and synthetic muscle promoters.

43. The vector of claim 40, wherein the promoter is selected from the group consisting of: CMV, RSV, SV40, β-actin, PGK, and EF1 promoters.

44. The vector of any one of claims 27-43, wherein the vector is a viral vector.

45. The vector of claim 44, wherein the viral vector is a lentivirus vector or herpes virus vector.

46. The vector of any one of claims 27-43, wherein the vector is a plasmid.

47. A pharmaceutical composition comprising a vector of any one of claims 27-46.

48. The pharmaceutical composition of claim 47, further comprising an adjuvant.

49. The pharmaceutical composition of claim 48, wherein the adjuvant is a CpG adjuvant.

50. A vaccine composition comprising a vector of any one of claims 27-46.

51. The vaccine composition of claim 50, further comprising an adjuvant.

52. The vaccine composition of claim 51, wherein the adjuvant is a CpG adjuvant.

53. A method of inducing at least partial immunity to a coronavirus in a subject, the method comprising administering to the subject a therapeutically effective amount of an AAV or AAV vector of any one of claims 1-20, a vector of any one of claims 27-46, a pharmaceutical composition of any one of claims 21-23 and 47-49, or a vaccine composition of any one of claims 24-26 and 50-52.

54. The method of claim 53, wherein the administering results in at least partial immunity to the coronavirus due to humoral immunity to the coronavirus.

55. The method of claim 53, wherein the administering results in robust T-cell mediated immunity to the coronavirus.

56. The method of claim 53, wherein the administering results in an increase in titer of antibodies that specifically bind to the SARS-CoV-2 polypeptide or the fragment thereof in the subject.

57. The method of any one of claims 53-56, wherein the administering results in a decrease in the rate of infection of the coronavirus in the subject.

58. The method of any one of claims 53-57, wherein the method further comprises administering an adjuvant to the subject.

59. The method of claim 58, wherein the adjuvant is a CpG adjuvant.

60. The method of any one of claims 53-59, wherein the subject has been identified as not having previously had a coronavirus infection.

61. The method of any one of claims 53-59, wherein, prior to the administering step, the subject has been identified as not having a significant titer of antibodies that bind specifically to the SARS-CoV-2 polypeptide of the fragment thereof.

62. The method of any one of claims 53-61, wherein the coronavirus is SARS-CoV-2.

63. The method of any one of claims 53-62, wherein the subject has been previously identified as having one or more medical conditions selected from the group consisting of: chronic lung disease, moderate asthma, severe asthma, heart conditions, diabetes, obesity, liver disease, chronic kidney disease, and a weakened or suppressed immune system.

64. The method of claim 63, wherein the subject having a weakened or suppressed immune system is a subject receiving a cancer treatment, a smoker, a subject who is a transplant recipient, a subject having HIV or AIDS, or a subject receiving a corticosteroid or any other immunosuppressant drug.

65. The method of claim 63, wherein the subject having a weakened or suppressed immune system is an elderly subject.


SEQUENCE LISTING (6 sequences)

SEQ ID NO: 1 - protein, 1273 aa, SARS-coronavirus-2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 588 aa, SARS-coronavirus-2
SVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - protein, 458 aa, SARS-coronavirus-2
SFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 4 - protein, 23 aa, SARS-coronavirus-2
WPWYIWLGFIAGLIAIVMVTIML

SEQ ID NO: 5 - protein, 419 aa, SARS-coronavirus-2
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 6 - protein, 15 aa, SARS-coronavirus-2
MFVFLVLLPLVSSQC